(19) United States
(12) Patent Application Publication
(10) Pub. No.: US 2019/0010173 A1
(43) Pub. Date: Jan. 10, 2019

(54) COMPOSITIONS AND METHODS FOR INHIBITION OF CATHEPSINS

(71) Applicants: Mateon Therapeutics, Inc., South San Francisco, CA (US); Baylor University, Waco, TX (US)

(72) Inventors: Kevin G. Pinney, Waco, TX (US); Mary Lynn Trawick, Waco, TX (US); Erica N. Parker, Waco, TX (US); David J. Chaplin, San Francisco, CA (US)

(73) Assignees: Mateon Therapeutics, Inc., South San Francisco, CA (US); Baylor University, Waco, TX (US)

(21) Appl. No.: 15/752,487

(22) PCT Filed: Aug. 12, 2016
(86) PCT No.: PCT/US16/46835

§ 371 Date: Feb. 13, 2018

Related U.S. Application Data
(60) Provisional application No. 62/205,500, filed on Aug. 14, 2015.

(51) Int. Cl. C07F 9/06; A61K 31/661; A61K 45/06

(52) U.S. Cl. CPC C07F 9/06; A61K 45/06; A61K 31/661

(57) ABSTRACT

This present disclosure is directed to compound of Formula I and methods of using these compounds in the treatment of conditions in which modulation of a cathepsin, particularly cathepsin L, cathepsin K, and/or cathepsin B, will be therapeutically useful. Formula I: or a solvate or pharmaceutically acceptable salt thereof. Each of R1-R10 are independently selected from the group consisting of: hydrogen, alkoxy, halo, hydroxy, phosphate, phosphate salts, disodium phosphate, diphosphate dimer, diphosphate dimer salt, and sodium diphosphate dimer with at least one of R1-R10 is a phosphate or diphosphate dimer group.

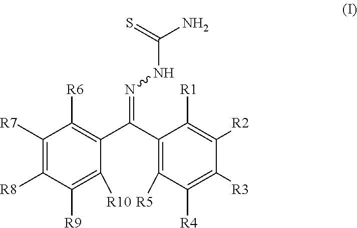

### CROSS REFERENCE TO RELATED APPLICATIONS

The present application claims priority under 35 U.S.C. 119 to U.S. provisional patent application Ser. No. 62/205,500 filed Aug. 14, 2016, titled “Compositions and Methods for Inhibition of Cathepsins”, the disclosure of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present invention relates to compounds and methods of using these compounds in the treatment of conditions in which modulation of the cathepsin activity, particularly cathepsin L or cathepsin K, is therapeutically useful.

### BACKGROUND

There are five classes of proteases including matrix metalloproteases (MMPs), cysteine proteases, serine proteases, aspartic proteases, and threonine proteases which catalyze the hydrolysis of peptide bonds. Due to their function in many disease states, including cancer and cardiovascular disease, proteases have become well-investigated therapeutic targets. Upregulation of MMPs is associated with cancer metastasis, consequently much research has been done to inhibit their activity. Since inhibitors of MMPs have failed to progress beyond clinical trials, interest in the other classes of proteases as therapeutic targets has grown significantly.

Cysteine protease cathepsins, members of the papain family, have recently been validated as an important enzymatic class to target in cancer research. In this family, there are eleven cathepsin enzymes known to date in humans: B, C, F, H, K, L, O, S, V, W, and X. Cathepsins are found in the highest concentration in cellular lysosomes, and during cancer progression they are secreted at an increased rate and degrade the extracellular matrix and basement membrane, which aid in cancer metastasis. Cathepsins B and L have been investigated extensively, due to their increased expression and activity in human and mouse tumors. Cathepsin K has also been the target of much research, due to its role in bone resorption and implications in osteoporosis.

Cathepsin inhibitors as drug candidates for the treatment of various diseases in the pharmaceutical pipeline include VBY-825 (Virobay), a pan cysteine protease inhibitor targeting the treatment of liver fibrosis, and Odanacatib (Merck), a cathepsin K inhibitor to suppress bone resorption in osteoporosis. Eli Lily was developing LY3000328 (Eli Lily) as a cathepsin S inhibitor targeting the treatment of abdominal aortic aneurysm.

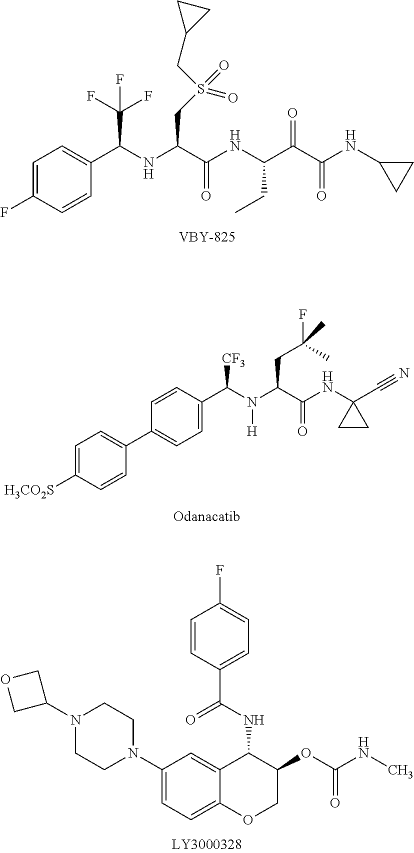

Cathepsin L also has a major function in intracellular lysosomal proteolysis, and in the degradation of the extracellular matrix (ECM) during the growth and metastasis of primary tumors. Despite the importance of cathepsin L in cancer metastasis and considerable interest in the enzyme as a target for synthesis of new potential anticancer agents, there are currently no clinical trials focused on testing inhibitors of cathepsin L in cancer metastasis. This is in contrast to the application of odanacatib to prevent bone loss in osteoporosis and cancer that has metastasized to bone. Odanacatib is a specific inhibitor of cathepsin K, an enzyme that is involved in degradation of the extracellular matrix proteins associated with bone resorption. Cathepsin K is a distinct enzyme in structure and function from that of cathepsin L. Small molecule inhibitors of cathepsin L have been synthesized incorporating different electrophilic moieties that can interact with the catalytic site residue Cys-25. Warheads which covalently bind with the Cys25 thiolate of cathepsin L include the epoxide in Clik 148 (I), the carbonyl of thiocarbazate II, the nitrile in the purine analogue III, the cyclic carbonyl in azepanone IV, the nitrile in the triazine analogue V, the α,β-unsaturated amide of gallinamide A (VI), and the aldehyde of the N-(1-naphthalenylsulfonyl) peptide derivative VII.

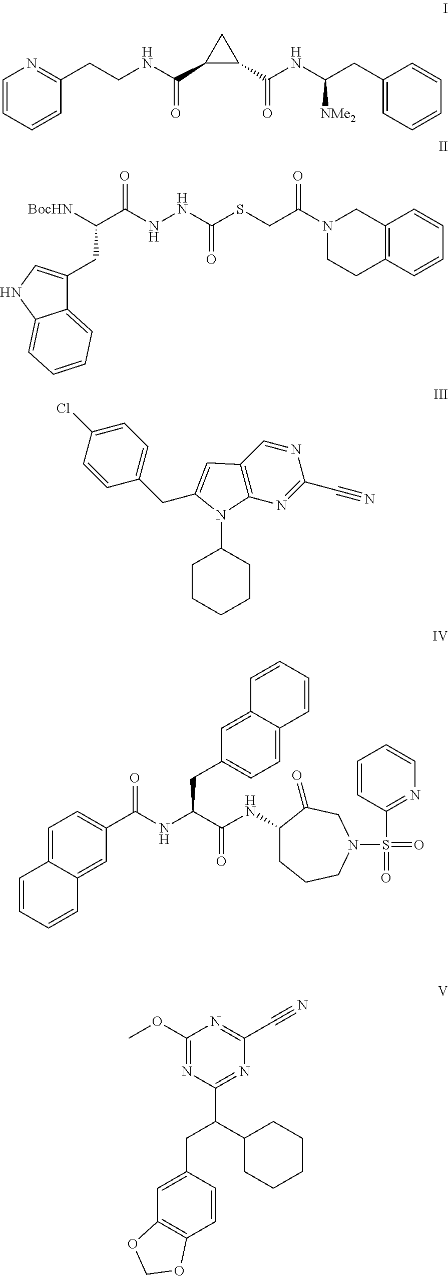

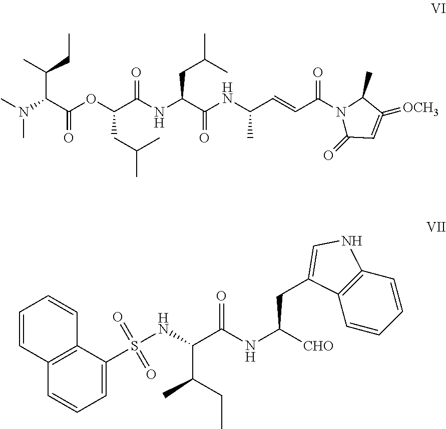

Cathepsin L also has been implicated in regulatory events relating to diabetes, immunological responses, degradation of the articular cartilage matrix, and other pathological processes (Chapman et al., 1997, Annu Rev Physiol 59:63-88; Turk and Guncar, 2003 Acta Crystallogr D Biol Crystallogr 59:203-213; Maehr et al., 2005, J Clin Invest 115:2934-2943; Vasiljeva et al., 2007, Curr Pharm Des 13:387-403), including osteoporosis and rheumatoid arthritis, (McGrath, 1999 Annu Rev Biophys Biomol Struct 28:181-204; Turk et al., 2001 EMBO J 20:4629-4633; Potts et al., 2004 Int J Exp Pathol 85:85-96; Schedel et al., 2004 Gene Ther 11:1040-1047). Further, inhibition of cathepsin L has also been shown to block Severe Acute Respiratory Syndrome (SARS) and Ebola pseudotype virus infection (Shah et al., 2010, Molecular Pharmacology 78(2):319-324).

U.S. Pat. No. 8,173,696 discloses ([(3-bromophenyl)-(3-hydroxyphenyl)-ketone] thiosemicarbazone), which has a formula of:

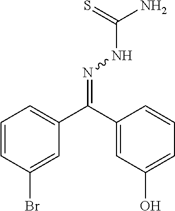 [SMILES: NC(=S)NN=C(c1cccc(O)c1)c1cccc(Br)c1]

[(3-bromophenyl)-(3-hydroxyphenyl)-ketone] thiosemicarbazone is a potent inhibitor of cysteine proteases cathepsin L and cathepsin K; however, this compound has poor aqueous solubility. A need exists for the development of a compound that has improved aqueous solubility and handling properties while still acting as a potent inhibitor of cysteine protease cathepsin L and other cathepsins.

### SUMMARY OF THE DISCLOSURE

This invention is directed to compounds and methods of using these compounds in the treatment of conditions in which modulation of a cathepsin, particularly cathepsin L or cathepsin K, will be therapeutically useful.

In general, in one embodiment, a compound of Formula I:

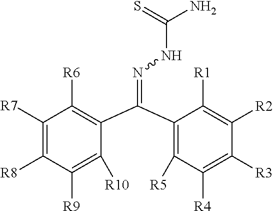

or a solvate or pharmaceutically acceptable salt thereof, wherein each of R1-R10 are independently selected from the group consisting of: hydrogen, alkoxy, halo, hydroxy, phosphate, phosphate salts, monosodium phosphate, disodium phosphate, dihydrogen phosphate, diphosphate dimer, diphosphate dimer salts, and sodium diphosphate dimers, and at least one of R1-R10 is a phosphate group or diphosphate dimer.

This and other embodiments can include one or more of the following features. At least one of R1-R5 can be a phosphate group. At least two of R1-R5 are phosphate groups. At least one of R6-R10 can be a phosphate group. At least two of R6-R10 can be phosphate groups. R2 and R4 can be phosphate groups. One of R1-R5 can be a diphosphate dimer group. One of R6-R10 can be a diphosphate dimer group. R3 can be a phosphate group. R1, R2, R4, and R5 can be hydrogen. R4 can be a phosphate group. R1-R3 and R5 can be hydrogen. The phosphate group can be disodium phosphate. The phosphate group can be monosodium phosphate. One of R1-R5 can be a diphosphate dimer group with one or more sodium atoms. The diphosphate dimer group can be a monosodium diphosphate dimer, disodium diphosphate dimer, or trisodium diphosphate dimer. At least one of R6-R10 can be a halo. R9 can be a halo. R6-R8 and R10 can be hydrogen. Halo can be bromine (Br). The compound can have the formula:

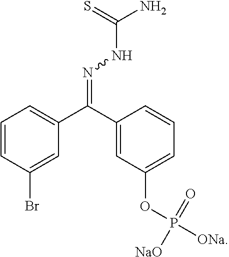

R9 can be bromine and R4 can be monosodium phosphate and R1-R3, R5-R8, and R10 can be hydrogen. R9 can be bromine and R4 can be phosphate and R1-R3, R5-R8, and R10 can be hydrogen. A method of inhibiting an activity of a cathepsin can include contacting the cathepsin with a compound in an amount of effective to inhibit an activity of the cathepsin. The cathepsin can be one or more of: cathepsin B, C, F, H, K, L, O, S, V, W, and X. A method of inhibiting an activity of a cathepsin can include contacting in vitro a cathepsin K or cathepsin L with a compound in an amount effective to inhibit an activity of the cathepsin. A method of inhibiting an activity of a cathepsin can include contacting in a patient a cathepsin with a compound in an amount of effective to inhibit an activity of the cathepsin. The method can further include administering a chemotherapy to the patient. The method can further include administering a radiation treatment to the patient. A method of inhibiting a neoplasm can include administering to a patient suffering from such neoplasm in an amount of a compound effective to treat the neoplasm. A method of providing an anti-metastatic therapy to a tumor can include administering to a patient in need of the anti-metastatic therapy a compound. A method of decreasing angiogenesis can include administering to a patient in need thereof a compound. Use of a compound can inhibit a neoplasm in a patient suffering from such a neoplasm. A pharmaceutical formulation can include a compound.

In general, in one embodiment, a method for synthesizing a compound includes providing a (3-Bromophenoxy)-tert-butyl-dimethyl-silane; reacting the (3-Bromophenoxy)-tert-butyl-dimethyl-silane with an n-butyllithium to form a (3-lithium-phenoxy)-tert-butyl-dimethyl-silane; and the reacting (3-lithium-phenoxy)-tert-butyl-dimethyl-silane with 3-Bromo-N-methoxy-N-methylbenzamide to form a [3-(t-Butyldimethylsilyl)oxyphenyl]-(3-bromophenyl) methanone.

This and other embodiments can include one or more of the following features. The method can further include reacting the [3-(t-Butyldimethylsilyl)oxyphenyl]-(3-bromophenyl) methanone with a thiosemicarbazide followed by desilylation to form a ([(3-bromophenyl)-(3-hydroxyphenyl)-ketone]thiosemicarbazone). The method can further include reacting the [3-(t-Butyldimethylsilyl)oxyphenyl]-(3-bromophenyl) methanone with a tetra-butyl ammonium fluoride trihydrate to form a (3-Bromophenyl)-(3-hydroxyphenyl) methanone. The method can further include reacting the (3-Bromophenyl)-(3-hydroxyphenyl) methanone with one or more of: carbon tetrachloride, 4-Dimethylaminopyridine, N,N-diisopropylethylamine, and dibenzyl phosphite to form a dibenzyl (3-(3-bromobenzoyl)phenyl) phosphate. The method can further include reacting the dibenzyl (3-(3-bromobenzoyl)phenyl) phosphate with a solution comprising HBr in AcOH or TMSBr to form a 3-(3-bromobenzoyl)phenyl dihydrogen phosphate. The method can further include reacting the 3-(3-bromobenzoyl)phenyl dihydrogen phosphate with a thiosemicarbazide followed by reacting with a sodium carbonate to form a 3-(3-bromobenzoyl)phenyl phosphate thiosemicarbazone.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a HPLC chromatogram of an alkaline phosphatase treated compound 27 (3-(3-bromobenzoyl)phenyl phosphate thiosemicarbazone) after 18 hours in comparison to compound 11 ([(3-bromophenyl)-(3-hydroxyphenyl)-ketone] thiosemicarbazone) in accordance with some embodiments.

FIG. 1B is a HPLC chromatogram of an alkaline phosphatase and 2% DMSO treated compound 27 (3-(3-bromobenzoyl)phenyl phosphate thiosemicarbazone) after 18 hours in comparison to compound 11 ([(3-bromophenyl)-(3-hydroxyphenyl)-ketone] thiosemicarbazone) in accordance with some embodiments.

### DETAILED DESCRIPTION

The present disclosure encompasses compounds having formula I and the compositions and methods using these compounds in the treatment of conditions in which inhibition of a cathepsin, particularly cathepsin L, cathepsin K, and/or cathepsin B, is therapeutically useful.

As used herein, the following definitions shall apply unless otherwise indicated.

The terms “cysteine protease” or “cysteine proteinase” or “cysteine peptidase” intend any enzyme of the sub-subclass EC 3.4.22, which consists of proteinases characterized by having a cysteine residue at the active site and by being irreversibly inhibited by sulfhydryl reagents such as iodoacetate. Mechanistically, in catalyzing the cleavage of a peptide amide bond, cysteine proteases form a covalent intermediate, called an acyl enzyme, that involves a cysteine and a histidine residue in the active site (Cys25 and His159 according to papain numbering, for example). Cysteine protease targets of particular interest in the present disclosure belong to the family C1 within the papain-like clan CA. Representative cysteine protease targets for the present disclosure include papain, cathepsin B (EC 3.4.22.1), cathepsin H (EC 3.4.22.16), cathepsin L (EC 3.4.22.15), cathepsin K, cathepsin S (EC 3.4.22.27), cruzain or cruzipain, rhodesain, brucipain, congopain, falcipain and CPB2.8 Delta CTE. Preferred cysteine protease targets of the present disclosure cleave substrate amino acid sequences -Phe-Arg-|-Xaa-, -Arg-Arg-|-Xaa-, -Val-Val-Arg-|-Xaa- or -Gly-Pro-Arg-|-Xaa-. Clan CA proteases are characterized by their sensitivity to the general cysteine protease inhibitor, E64 (L-trans-epoxysuccinyl-leucyl-amido (4-guanidino) butane) and by having substrate specificity defined by the S₂ pocket.

Cysteine proteases inhibited by the compounds of the present disclosure can be “cathepsin L-like” or “cathepsin B-like.” A cathepsin L-like cysteine protease shares structural and functional similarity with a mammalian cathepsin L, and comprises a “ERFNIN” motif (Sajid and McKerrow, supra). Cathepsin L-like cysteine proteases prefer as a substrate the dipeptide sequence -Phe-Arg-|-Xaa-. Representative cathepsin L-like cysteine proteases include cathepsin L, cathepsin K, cathepsin S, cruzain, rhodesain and congopain, T. cruzi-L, T. rangeli-L, T. congolense-L, T. brucei-L, P. falciparum-L1, P. falciparum-L2, P. falciparum-L3, P. vivax-L1, P. cynomolgi-L1, P. vinckei-L and L. major-L. A cathepsin B-like cysteine protease shares structural and functional similarity with a mammalian cathepsin B, and comprises an “occluding loop” (Sajid and McKerrow, supra). Cathepsin B-like cysteine proteases cleave as a substrate the dipeptide sequences -Arg-Arg-|-Xaa- and -Phe-Arg-|-Xaa-. Representative cathepsin B-like proteases include cathepsin B, T. cruzi-B, L. mexicana-B and L. major-B.

“Inhibitors” or “inhibition” of cysteine proteases refers to inhibitory compounds identified using in vitro and in vivo assays for cysteine protease function. In particular, inhibitors refer to compounds that decrease or obliterate the catalytic function of the target cysteine protease, thereby interfering with or preventing the infectious life cycle of a parasite or the migratory capacity of a cancer cell or an inflammatory cell. In vitro assays evaluate the capacity of a compound to inhibit the ability of a target cysteine to catalyze the cleavage of a test substrate. Cellular assays evaluate the ability of a compound to interfere with the migration of a cancer or inflammatory cell or the infectious life cycle of a parasite ex vivo, while not exhibiting toxicity against the host cell. Cellular assays measure the survival of a parasite-infected cell in culture. Preferred inhibitors allow for extended survival of an infected cell, either by delaying the life cycle of the parasite, or by killing the parasite. In vivo assays evaluate the efficacy of test compounds to prevent or ameliorate disease symptoms, such as those associated with parasitic infection, cancer invasion or growth, or inflammatory cell migration. Inhibitors are compounds that eliminate or diminish the catalytic function of a cysteine protease. Further, preferred inhibitors delay, interfere with, prevent or eliminate the completion of the infectious life cycle of a parasite or the migratory ability of a cancer cell or an inflammation cell. Additionally, preferred inhibitors prevent or diminish a parasitic infection in an individual or the migration of cancer cells or inflammatory cells in an individual, thereby preventing or ameliorating the pathogenic symptoms associated with such infections or the migration of rogue cells.

To examine the extent of inhibition, samples, assays, cultures or test subjects comprising a target cysteine protease are treated with a potential inhibitor compound and are compared to negative control samples without the test compound, and positive control samples, treated with a compound known to inhibit the target cysteine protease. Negative control samples (not treated with a test compound), are assigned a relative cysteine protease activity level of 100%. Inhibition of a cysteine protease is achieved when the cysteine protease activity relative to the control is about 90%, preferably 75% or 50%, more preferably 25-0%.

An amount of compound that inhibits a cysteine protease, as described above, is an amount sufficient to inhibit a “cysteine protease,” or a “cysteine protease inhibiting amount” of compound, thereby preventing or treating a parasitic infection, inflammation, or cancer invasion or growth in an individual.

The term “IC₅₀” refers to the concentration of compound that results in half-maximal inhibition of enzyme.

“Alkyl” refers to monovalent saturated aliphatic hydrocarbon groups having from 1 to 10 carbon atoms and preferably 1 to 6 carbon atoms. This term includes, by way of example, linear and branched hydrocarbon groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Amino” refers to the group —NH₂.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like), provided that the point of attachment is through an atom of the aromatic aryl group. Preferred aryl groups include phenyl and naphthyl.

“Alkenyl” refers to straight chain or branched hydrocarbon groups having from 2 to 6 carbon atoms and preferably 2 to 4 carbon atoms and having at least 1 and preferably from 1 to 2 sites of double bond unsaturation. Such groups are exemplified, for example, bi-vinyl, allyl, and but-3-en-1-yl. Included within this term are the cis and trans isomers or mixtures of these isomers.

“Alkynyl” refers to straight or branched monovalent hydrocarbon groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like.

“Halo” or “halogen” refers to fluoro, chloro, bromo, and iodo and is preferably fluoro, bromo, or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen, and sulfur within the ring. Such heteroaryl groups can have a single ring (e.g., pyridinyl, imidazolyl or furyl) or multiple condensed rings (e.g., indolizinyl, quinolinyl, benzimidazolyl or benzothienyl), wherein the condensed rings may or may not be aromatic and/or contain a heteroatom, provided that the point of attachment is through an atom of the aromatic heteroaryl group. In one implementation, the nitrogen and/or sulfur ring atom(s) of the heteroaryl group are optionally oxidized to provide for the N-oxide (N—O), sulfinyl, or sulfonyl moieties. Preferred heteroaryls include pyridinyl, pyrrolyl, indolyl, thiophenyl, and furanyl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In one implementation, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Nitro” refers to the group —NO₂.

“Nitroso” refers to the group —NO.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

The term “substituted,” when used to modify a specified group or radical, means that one or more hydrogen atoms of the specified group or radical are each, independently of one another, replaced with the same or different substituent groups as defined below.

Substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O)₂(M+)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means e.g. that one of the counter ions for such divalent alkali earth ions can be an ionized form of a compound of the present disclosure and the other a typical counter ion such as chloride, or two ionized compounds of the present disclosure can serve as counter ions for such divalent alkali earth ions, or a doubly ionized compound of the present disclosure can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In a preferred implementation, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to -substituted aryl-(substituted aryl)-substituted aryl.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

“Patient” refers to human and non-human animals, especially mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders and neoplasms, a pharmaceutically or therapeutically effective amount comprises an amount sufficient to, among other things, cause the tumor to shrink or decrease the growth rate of the tumor.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water.

Similarly, it is understood that the above definitions are not intended to include impermissible substitution patterns (e.g., methyl substituted with 5 fluoro groups). Such impermissible substitution patterns are easily recognized by a person having ordinary skill in the art.

### Chemical Compounds

The present disclosure provides novel thiosemicarbazone compounds and methods of making the compound and methods of using these compounds in the treatment of conditions in which inhibition of a cathepsin, particularly cathepsin L, cathepsin K, and/or cathepsin B, is therapeutically useful. These conditions include, but are not limited to, neoplasms, osteoporosis, protozoal parasite infection and viral infections. In some embodiments any of the compounds disclosed herein are used to cause an anti-metastatic response in a tumor. In some embodiments any of the compounds disclosed herein are used to decrease angiogenesis. In some embodiments the compounds disclosed herein can be used in combination with conventional chemotherapy treatments such as chemotherapy drugs and/or radiation. Given the severity of and suffering caused by these conditions, it is vital that new treatments are developed to treat these conditions.

U.S. Pat. No. 8,877,967 discloses compounds for inhibiting cathepsins. U.S. Pat. No. 8,173,696 discloses ([(3-bromophenyl)-(3-hydroxyphenyl)-ketone] thiosemicarbazone):

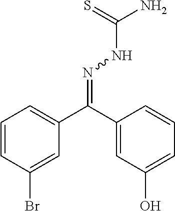 [SMILES: NC(=S)NN=C(c1cccc(O)c1)c1cccc(Br)c1]

As noted above ([(3-bromophenyl)-(3-hydroxyphenyl)-ketone] thiosemicarbazone) has poor water solubility. It is desirable to modify the compound to improve the handling properties such as the water solubility while still maintaining good activity. Disclosed herein are phosphate prodrugs having a good biological activity and improved handling properties.

In some embodiments, a compound having the Formula I or a solvate, or an isomer, or a pharmaceutically acceptable salt thereof is provided. Formula I:

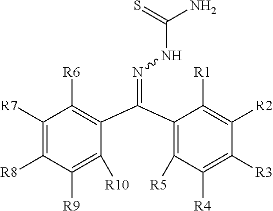

Each of R1-R10 in Formula I can be independently selected from the group consisting of: hydrogen, alkoxy, halo, hydroxy, phosphate, phosphate salts, disodium phosphate, dihydrogen phosphate, diphosphate dimers, diphosphate dimer salts, and sodium diphosphate dimers. In some embodiments at least one of R1-R10 is a phosphate group. In some embodiments two or more of R1-R10 are phosphate groups. In some embodiments two of R1-R5 are phosphate groups. In some embodiments two of R6-R10 are phosphate groups. In any of the compounds described herein the phosphate group can be replaced with a diphosphate dimer group (e.g. a diphosphate dimer having a PO₃PO₄(-3) formula).

In some embodiments at least one of R6-R10 is a halo. In some embodiments there is only one halo in R6-R10 with the remainder being hydrogen. In some embodiments R9 is a halo and R6-R8 and R10 are hydrogen. In a preferred embodiment R9 is bromine and R6-R8 and R10 are hydrogen.

In some embodiments at least one of R1-R5 is a phosphate group. In some embodiments at least one of R6-R10 is a phosphate group. In some embodiments at least two of R1-R10 is a phosphate group. In some embodiments only one of R1-R5 is a phosphate or diphosphate dimer group. In some embodiments only one of R1-R5 is a phosphate or diphosphate dimer group and the remainder of R1-R5 are hydrogen. In some embodiments the phosphate or diphosphate dimer group comprises an element selected from group I of the periodic table. In some embodiments the phosphate or diphosphate dimer group includes one or more monovalent metal cations. In some embodiments the phosphate group is disodium phosphate. In some embodiments the phosphate group is monosodium phosphate. In some embodiments the phosphate group is dihydrogen phosphate. In some embodiments the phosphate group is a diphosphate dimer group including one or more sodium atoms. In some embodiments R4 is a phosphate or diphosphate dimer. In some embodiments R4 is a phosphate or diphosphate dimer with R1-R3 and R5 hydrogen. In some embodiments R4 is disodium phosphate or diphosphate dimer including one or more sodium atoms. In some embodiments R4 is disodium phosphate or diphosphate dimer including one or more sodium atoms with R1-R3 and R5 hydrogen. Examples of diphosphate dimer groups including one or more sodium atoms include monosodium diphosphate dimer, disodium diphosphate dimer, and trisodium diphosphate dimer.

Any of the disodium phosphates groups described herein, including disodium phosphate, diphosphate dimers, diphosphate dimer salts, and sodium diphosphate dimers, can include a mixture of disodium phosphates and mono-sodium phosphates. For example, some monosodium phosphate groups and dihydrogen phosphate groups can be present depending on the solvent type, pH, and other properties of the solvent.

In some embodiments of Formula I, R4 is disodium phosphate and R9 is bromine, resulting in the compound:

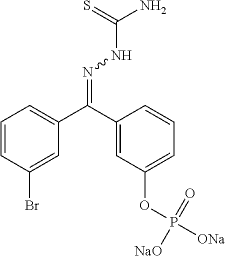

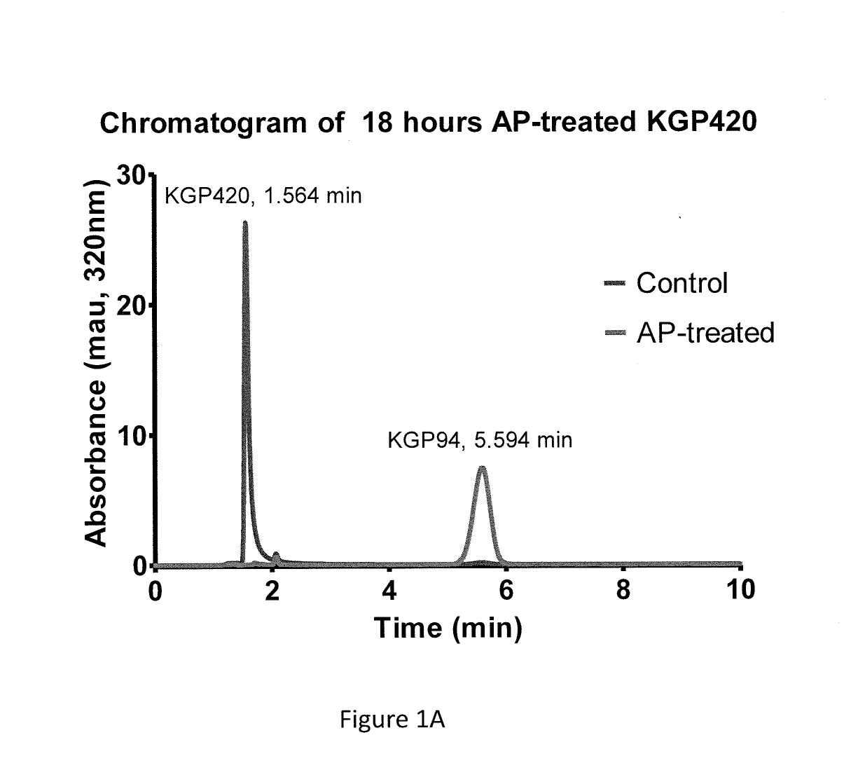

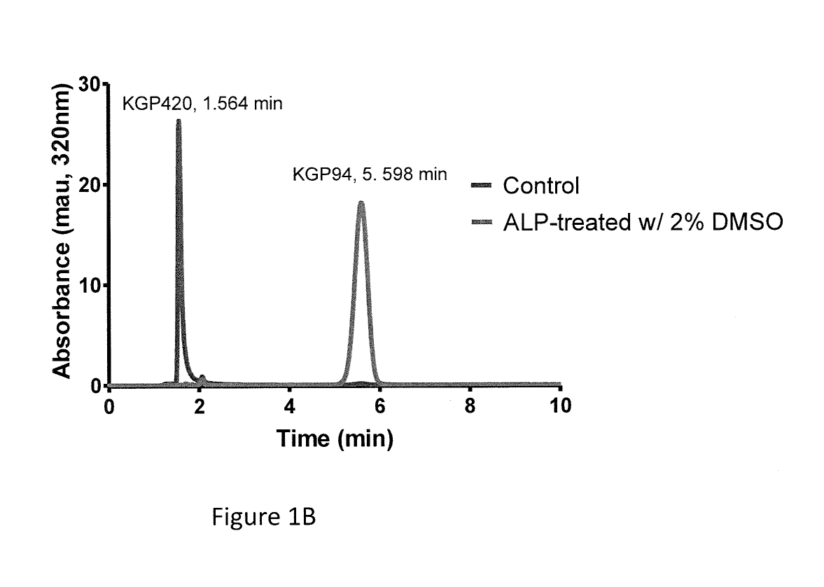

In some embodiments the compounds of Formula I include phosphate prodrugs. The compounds of Formula I can include one or more phosphate group that can undergo dephosphorylation to remove the phosphate group in vivo. For example, hydrolysis, such as enzymatically driven hydrolysis, can remove the phosphate group from the compound in vivo to become active with respect to cathepsins. For example, compound 27 has good aqueous solubility and can hydrolyze in vivo to become biologically active as discussed below with respect to FIGS. 1A-1B.

In some embodiments any compound can be selected from Table 1, or a solvate, tautomer, stereoisomer, and/or pharmaceutically acceptable salt thereof. The compounds covered by Formula I can include multiple stereoisomers. For example, the stereoisomers can included E and Z geometrical isomers that can be present in varying ratios depending on the compound and surrounding medium. Any of the compounds and formulations disclosed herein can include multiple geometric isomers.

TABLE 1

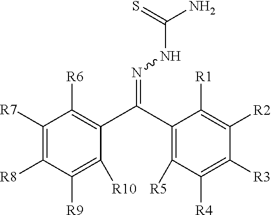

| | R1 | R2 | R3 | R4 | R5 | R6 | R7 | R8 | R9 | R10 |
|---|---|---|---|---|---|---|---|---|---|---|
| 11 | H | H | H | OH | H | H | H | H | Br | H |
| 12 | H | OH | H | Br | H | H | OH | H | Br | H |
| 13 | H | H | H | OH | H | H | Br | H | Br | H |
| 19 | H | —OCH₃ | H | OCH₃ | H | H | H | H | Br | H |
| 20 | H | OCH₃ | H | OCH₃ | H | H | Br | H | Br | H |
| 21 | H | OH | H | OH | H | H | H | H | Br | H |
| 22 | H | OH | H | OH | H | H | Br | H | Br | H |
| 27 | H | H | H | Na₂PO₄ | H | H | H | H | Br | H |
| 40 | H | OH | H | Br | H | H | H | H | Br | H |
| 41 | H | H | OH | Br | H | H | H | H | Br | H |
| 42 | H | H | H | Br | OH | H | H | H | Br | H |
| 43 | H | H | OH | Br | H | H | H | OH | Br | H |
| 44 | H | H | H | Br | OH | OH | H | H | Br | H |
| 45 | H | OH | H | Br | H | H | OH | H | Br | H |
| 46 | Na₂PO₄ | H | H | H | H | H | H | H | Br | H |
| 47 | H | Na₂PO₄ | H | H | H | H | H | H | Br | H |
| 48 | H | H | Na₂PO₄ | H | H | H | H | H | Br | H |
| 49 | H | H | H | H | Na₂PO₄ | H | H | H | Br | H |
| 50 | H | H | H | OH | H | Na₂PO₄ | H | H | H | H |
| 51 | H | H | H | OH | H | H | Na₂PO₄ | H | H | H |
| 52 | H | H | H | OH | H | H | H | Na₂PO₄ | H | H |
| 53 | H | H | H | OH | H | H | H | H | Na₂PO₄ | H |
| 54 | H | H | H | OH | H | H | H | H | H | Na₂PO₄ |
| 55 | H | Na₂PO₄ | H | Na₂PO₄ | H | H | H | H | Br | H |
| 56 | H | Na₂PO₄ | H | Br | H | H | Na₂PO₄ | H | Br | H |

*Disodium phosphate (Na₂PO₄) in compounds 27 and 46-54 can be replaced by monosodium phosphate, a dihydrogen phosphate, or any of the disphosphate dimer groups disclosed herein.

In some embodiments two of R1-R5 are phosphate groups. In some embodiments R2 and R4 are disodium phosphate and R9 is bromine. In some embodiments the compound has the following structure:

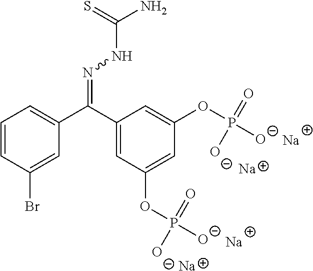 [SMILES: NC(=S)NN=C(c1cccc(Br)c1)c1cc(OP(=O)([O-])[O-])cc(OP(=O)([O-])[O-])c1 ; [Na+] ; [Na+] ; [Na+] ; [Na+]]

In some embodiments one of R1-R5 is a phosphate group and one of R6-R10 is a phosphate group. In some embodiments one of R1-R5 is a bromine and one of R6-R10 is bromine. In some embodiments R2 and R7 are disodium phosphate and R4 and R9 are bromine. In some embodiments the compound has the following structure:

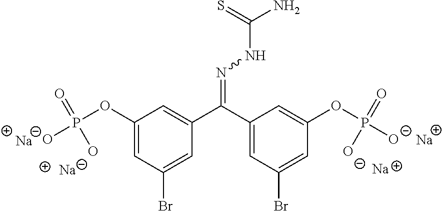

In some embodiments R4 is a diphosphate dimer group an R9 is bromine. For example R4 is trisodium diphosphate dimer and R9 is bromine thereby producing the following structure:

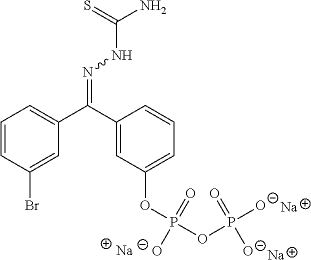

In some embodiments the compound can be a dimer of Formula I. For example two of Formula I can be connected by a diphosphate dimer group. For example two of compound 27 can be connected through the phosphate groups making a diphosphate dimer between the phenyl rings.

The biological activity for a number of these compounds has been studied with respect to cathepsin L, cathepsin B, and/or cathepsin K.

Table 2 shows the inhibitory activity for benzoylbenzophenone thiosemicarbazone analogues.

TABLE 2

| | IC₅₀^{a} Values (nM) | | |
|---|---|---|---|
| Cmpd | Cat L | Cat B | Cat K |
| 11 | 189 | >10000 | 53 |
| 12 | >10000 | >10000 | 369 |
| 13 | 202 | >10000 | NC |
| 19 | >10000 | >10000 | >10000 |
| 20 | >10000 | >10000 | 105 |
| 21 | ~10000 | >10000 | >10000 |
| 22 | >10000 | >10000 | 203 |
| 27 | >10000 | ND* | ND |

^{a}These values are averages of a minimum of a triplicate of experiments. Each assay for Cat. L and Cat. B utilized 2% DMSO with a 5 min pre-incubation period. For Cat. K a 4% DMSO was used with a 5 min pre-incubation period. Some assays for Compound 27 also included the use of a surfactant like 0.1% Tween or 0.1% CHAPS (3-[(3-Cholamidopropyl)dimethylammonio]-1-propanesulfonate hydrate).
ND = not determined.

The compounds disclosed herein had varying activity against cathepsin L. In some compounds it appears that the presence of two m-hydroxyl or two m-dimethoxyl substituents may impair inhibitory activity against cathepsin L. In some embodiments only one of R1-R5 is substituted with the variable groups disclosed herein. In some embodiments only one of R6-R10 is substituted with a halo group. In some embodiments one of R1-R5 is a halo and only one of R6-R10 is a phosphate group with the remaining groups being hydrogen.

In some embodiments the compounds disclosed herein can have activity against cathepsin L and K. Activity against both cathepsin L and K can be beneficial. For example, compound 11 has a cathepsin K activity of 53 nM. After dephosphorylation compound 27 is expected to have a similar in vitro activity to compound 11.

FIGS. 1A-1B are HPLC chromatograms of compound 27 treated with alkaline phosphatase and alkaline phosphatase and 2% DMSO, respectively, after 18 hours. The control in FIGS. 1A-1B illustrates the absorbance peak corresponding to compound 27 (also known as KGP420) at 1.564 minutes. The absorbance for compound 27 treated in an alkaline phosphatase solution exhibited a peak at 5.594 minutes (FIG. 1A), which corresponds to compound 11. The absorbance for compound 27 treated in a 2% DMSO alkaline phosphatase solution exhibited a peak at 5.598 minutes (FIG. 1B), which corresponds to compound 11. FIGS. 1A-I B show that compound 27, after enzymatic cleavage of the phosphate, has a structure similar to compound 11. Enzymatic cleavage of compound 27 yielded compound 11 which inhibited cathepsin L by 88% at 10 μM. Compound 27 has significantly higher aqueous solubility than compound 11. In addition it appears that, compound 27, prior to enzymatic cleavage of the phosphate group, exhibited low cathepsin activity as shown in Table 2. Thus, the activity of compound 27 (and other phosphate prodrugs disclosed herein) can be triggered by dephosphorylation, such as by enzymatically cleaving the phosphate promoiety or phosphate group.

Table 3 illustrates human umbilical vein endothelial cell (HUVEC) data for the cytotoxicity of compounds 11 and 27.

TABLE 3

| | Compound 11 | Compound 27 |
|---|---|---|
| Cytotoxicity GI₅₀ (μM) | 26.9 | 20.2 |

The stability of compound 27 was evaluated in aqueous solution. Compound 27 underwent very minor spontaneous hydrolysis over 48 h incubation at 37° C. in 10 mM glycine buffer solution (pH 8.6) without alkaline phosphatase (ALP) (FIG. 1A). Additionally, compound 27 was not hydrolyzed when stored in water at 4° C. for one week. Enzymatic cleavage of compound 27 occurred with nearly 100% conversion to the anticipated parent drug compound 11 when treated with 1 unit of ALP over the course of 48 hours (FIG. 1B). Enzymatic cleavage of compound 27 yielded compound 11 which inhibited cathepsin L by 88% at 10 μM. Compound 27 post enzymatic cleavage was active against cathepsin L. Compound 27 also appears to have a high stability.

### Synthesis of Select Compounds Disclosed Herein

Improved synthesis schemes for the compounds described herein are also disclosed in the present application. Synthesis of the phosphate prodrug compounds disclosed herein presented several challenges discussed below. In general the synthesis methods include phosphorylating a scaffold, installing a thiosemicarbazone onto the scaffold, deprotection of the phosphate, followed by salt formation.

Structure-activity relationship (SAR) studies of thiosemicarbazone inhibitors based on the benzophenone scaffold highlight the importance of the 3-bromophenyl moiety. The extended series of inhibitors incorporate one or more of m-hydroxy, m-methoxy, and m-bromo substituents onto the benzophenone molecular scaffolds. A number of challenges were presented in the synthesis of the compounds described herein, including the formation of trace impurities and undesirable reactions. For example, the previously employed synthesis route that resulted in trace impurities is discussed below with respect to scheme 1.

Additional challenges were also presented by attempting phosphorylation of hydroxybenzophenone thiosemicarbazones. For example, phosphorylation onto a hydroxybenzophenone thiosemicarbazone resulted in multiple products along with instances of an extra benzyl group attached to the resulting product. Consequently, synthesis methods were explored for forming the compounds disclosed herein by first phosphorylation of the ketone followed by installing the thiosemicarbazone.

The initial synthetic route to compound 11 (also known as KGP94) is shown in Scheme 1. Scheme 1 utilized the addition of 3-bromophenyl magnesium bromide to the corresponding Weinreb amide to afford (3-bromophenyl)-(3-hydroxyphenyl)-methanone which was condensed with thiosemicarbazide followed by deprotection of the silyl ether lead to the formation of compound 11. However, HPLC analysis of the final products revealed that a trace amount of bromine had been replaced by hydrogen. Replacement of bromine by hydrogen likely occurred during the metal-halogen exchange reaction where excess magnesium was used to prepare benzophenone (3-bromophenyl)-(3-hydroxyphenyl)-methanone.

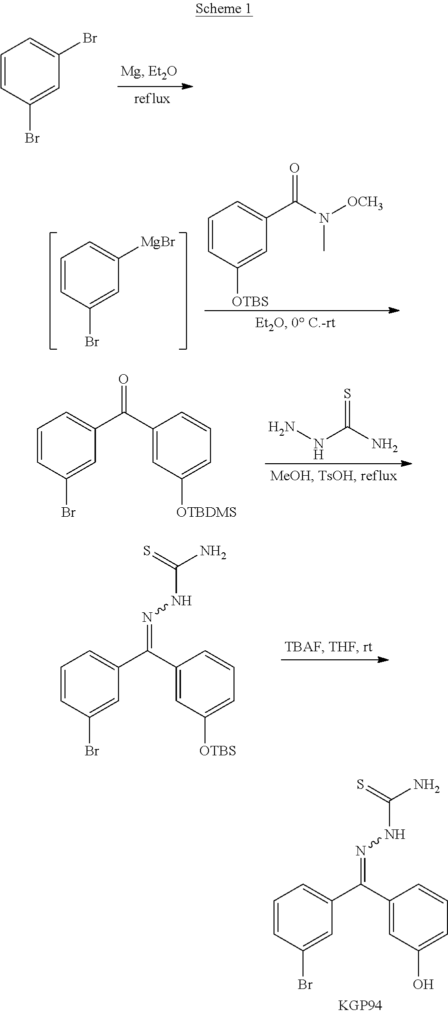

In an effort to avoid these trace impurities, the compounds disclosed herein were synthesized utilizing a revised route, illustrated in Scheme 2. Instead of using 1,3-dibromobenzene as the precursor to the organometallic reagent for the synthesis of compound 11, a protected m-bromophenol 1 was reacted with n-butyllithium to form the intermediate organolithium reagent which was reacted with Weinreb amide 4 to afford the desired functionalized benzophenone 7. Benzophenones 8 and 10 were synthesized in a similar manner by reacting the appropriately substituted aromatic ring with n-butyllithium followed by the addition of Weinreb amide 5 to afford ketone 8 or the addition of aldehyde 6 followed by oxidation with PCC to form ketone 10. Condensation of benzophenones 7, 8, and 10 (separately) with thiosemicarbazide followed by desilylation with TBAF afforded compound 11 and final thiosemicarbazone analogues of compound 11, including compounds 12 and 13. HPLC analysis showed no trace amount of impurities in which bromine was replaced by hydrogen in the final products.

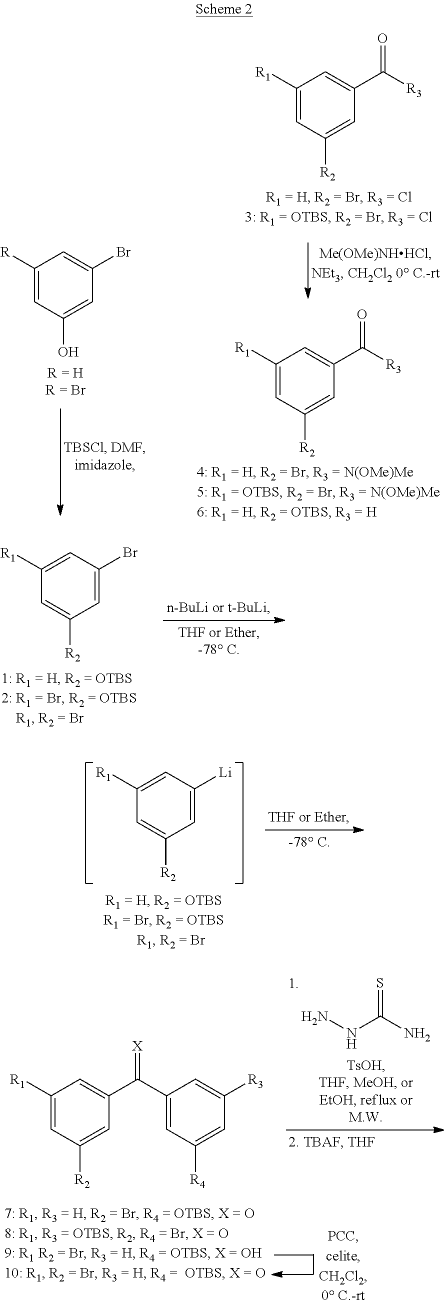

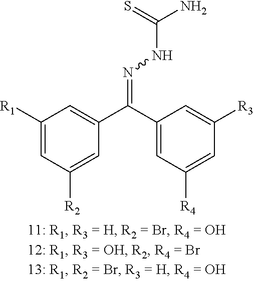

Scheme 3 illustrates the synthesis of dimethylresorcinol and resorcinol analogues. The synthesis of dimethylresorcinol and resorcinol analogues utilized commercially available 1-bromo-3,5-dimethoxy benzene as a starting material to form an intermediate organolithium reagent which was reacted with Weinreb 4 or 14 to form ketones 15 and 16, respectively. Demethylation of 3,5-dimethoxy benzophenones 15 and 16 with boron tribromide afforded 3,5-dihydroxy benzophenones 17 and 18. Condensation of benzophenones 15-18 with thiosemicarbazide under microwave irradiation afforded final compounds 19-22.

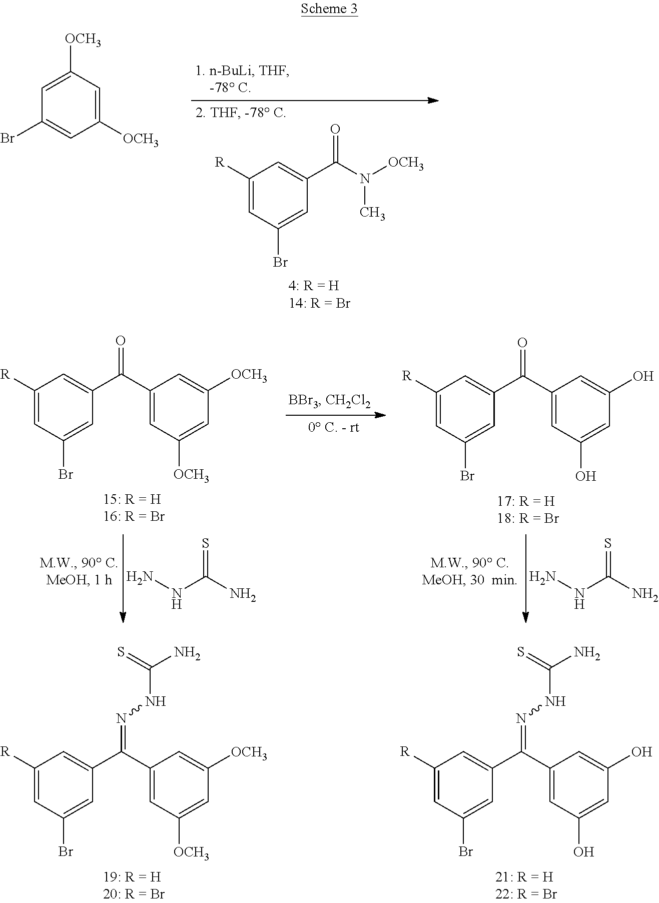

In order to increase the solubility and bioavailability of compound 11, prodrug derivatization was attempted by phosphorylation of the phenol. The (3-Bromophenyl)-(3-hydroxyphenyl) methanone 23 was phosphorylated with dibenzyl chlorophosphate (prepared in situ) to afford the dibenzyl phosphate ester 24 followed by deprotection of the benzyl groups with 33% HBr in AcOH, which generated phosphoric acid ester 25. Successful completion of the synthesis of the phosphate salt of benzophenone thiosemicarbazone 27 was accomplished by the condensation of phosphoric acid ester 25 with thiosemicarbazide to afford benzophenone thiosemicarbazone 26 which was subsequently reacted with sodium carbonate to generate the disodium phosphate salt 27.

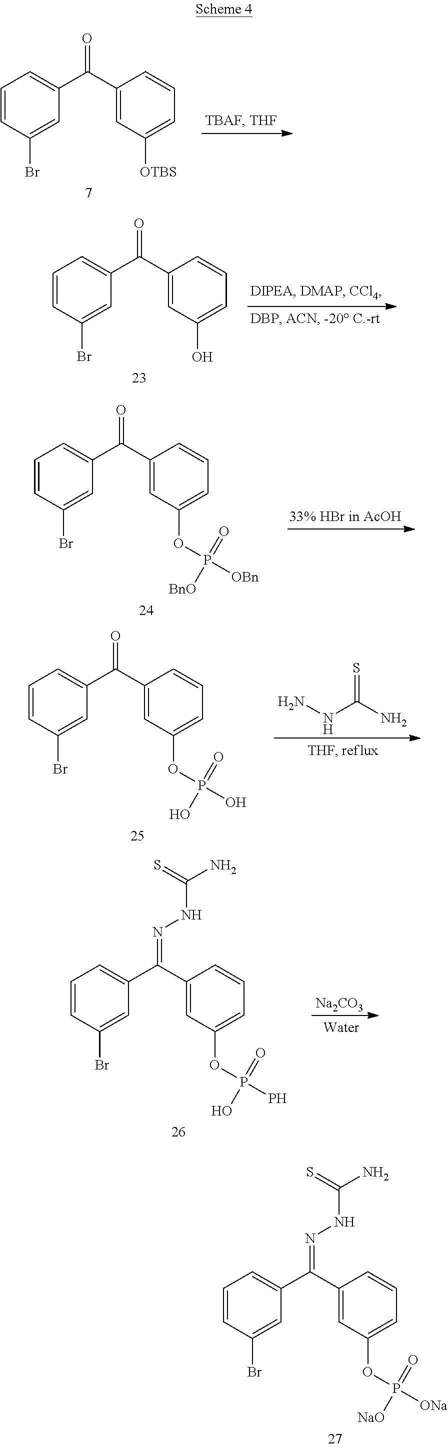

As noted above, challenges were encountered with various methods for deprotection of the phosphate by removal of the benzyl groups for dibenzyl(3-(3-bromobenzoyl)phenyl) phosphate and dibenzyl(3-benzoylphenyl) phosphate.

For example, the reduction of dibenzyl(3-(3-bromobenzoyl)phenyl) phosphate or dibenzyl(3-benzoylphenyl) phosphate with Pd/C resulted in the formation of multiple products and longer reaction times. Instead deprotection of dibenzyl(3-(3-bromobenzoyl)phenyl) phosphate or dibenzyl(3-benzoylphenyl) phosphate was done using either TMSBr or 33% HBr in AcOH to yield the desired corresponding phosphoric acid product. The use of 33% HBr in AcOH was preferred due to the ease of carrying out the reaction. Condensation of the thiosemicarbazide with 3-(3-bromobenzoyl)phenyl dihydrogen phosphate was followed by salt formation with sodium carbonate to yield the desired product as illustrated in scheme 4.

In some embodiments methods for synthesizing compound 11 are provided. The methods can include providing (3-Bromophenoxy)-tert-butyl-dimethyl-silane, reacting (3-Bromophenoxy)-tert-butyl-dimethyl-silane with n-butyllithium to form (3-lithium-phenoxy)-tert-butyl-dimethyl-silane, and reacting (3-lithium-phenoxy)-tert-butyl-dimethyl-silane with 3-Bromo-N-methoxy-N-methylbenzamide to form [3-(t-Butyldimethylsilyl)oxyphenyl]-(3-bromophenyl) methanone. [3-(t-Butyldimethylsilyl)oxyphenyl]-(3-bromophenyl) methanone can then be reacted with thiosemicarbazide followed by desilylation to form compound 11.

In some embodiments methods for synthesizing compound 27 are provided. The methods can include providing (3-Bromophenoxy)-tert-butyl-dimethyl-silane, reacting (3-Bromophenoxy)-tert-butyl-dimethyl-silane with n-butyllithium to form (3-lithium-phenoxy)-tert-butyl-dimethyl-silane, and reacting (3-lithium-phenoxy)-tert-butyl-dimethyl-silane with 3-Bromo-N-methoxy-N-methylbenzamide to form [3-(t-Butyldimethylsilyl)oxyphenyl]-(3-bromophenyl) methanone. [3-(t-Butyldimethylsilyl)oxyphenyl]-(3-bromophenyl) methanone can be further reacted with tetra-butyl ammonium fluoride trihydrate to form (3-Bromophenyl)-(3-hydroxyphenyl) methanone. (3-Bromophenyl)-(3-hydroxyphenyl) methanone can be reacted with one or more of: carbon tetrachloride, 4-Dimethylaminopyridine, N,N-diisopropylethylamine, and dibenzyl phosphite to form dibenzyl (3-(3-bromobenzoyl)phenyl) phosphate. Dibenzyl (3-(3-bromobenzoyl)phenyl) phosphate can be reacted with 33% HBr in AcOH or TMSBr to form 3-(3-bromobenzoyl)phenyl dihydrogen phosphate. 3-(3-bromobenzoyl)phenyl dihydrogen phosphate can be reacted with thiosemicarbazide followed by reacting with sodium carbonate to form compound 27.

In some embodiments a ketone with two benzyl rings that corresponds to any of the compounds in Formula I can be phosphorylated followed by condensing with a thiosemicarbazone and subsequent reduction to form salt of any of the compounds in Formula I.

Scheme 5 illustrates additional synthesis schemes that can be used to make a number of the compounds described herein in accordance with some embodiments.

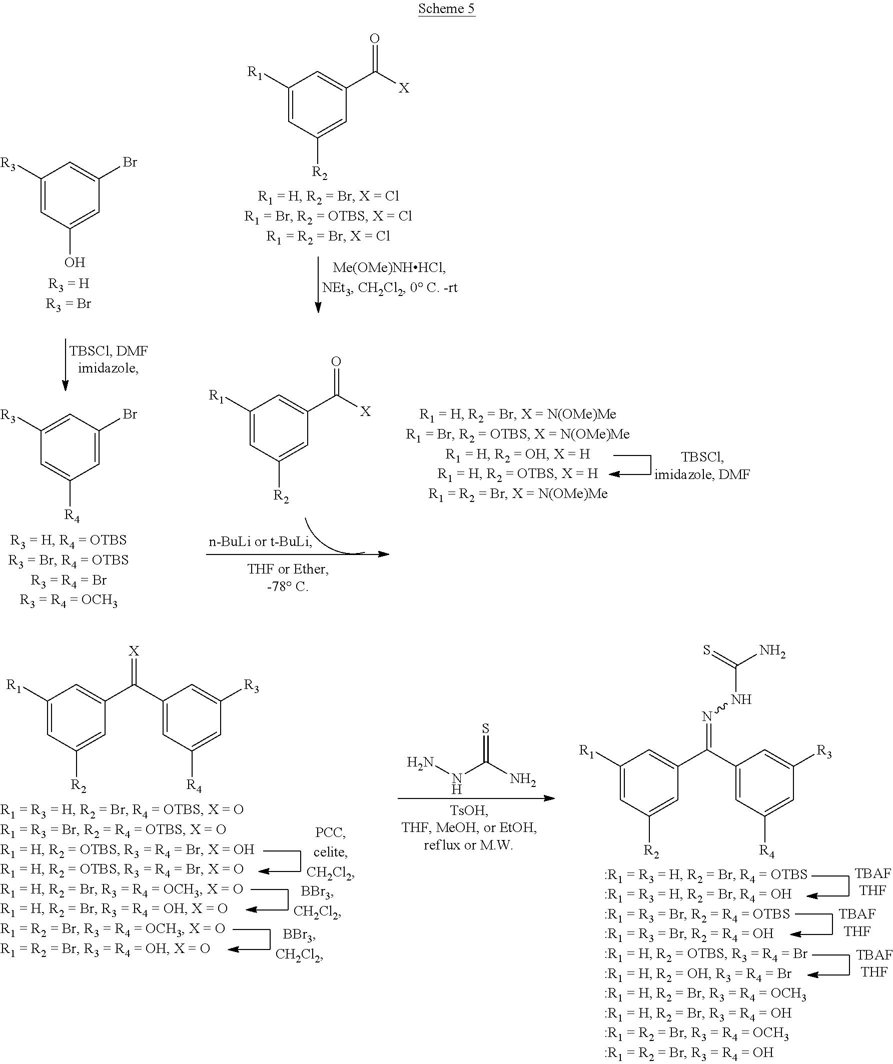

Additional specific examples of methods for synthesizing many variations of the compounds disclosed herein are provided in the Examples.

Depending upon the nature of the various substituents, the thiosemicarbazone compounds disclosed herein can be in the form of salts. Such salts include salts suitable for pharmaceutical uses (“pharmaceutically-acceptable salts”), salts suitable for veterinary uses, etc. Such salts can be derived from acids or bases, as is well-known in the art.

In one implementation, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4 chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion) or coordinates with an organic base (e.g., ethanolamine, diethanolamine, triethanolamine, N-methylglucamine, morpholine, piperidine, dimethylamine, diethylamine, triethylamine, ammonia, etc.).

The thiosemicarbazone compounds and salts thereof may also be in the form of hydrates, solvates and N-oxides, as are well-known in the art.

In another implementation, this disclosure provides a compound found in Table 1, or stereoisomer, tautomer, solvate, or pharmaceutically acceptable salt thereof.

The compounds of the present disclosure are surprisingly potent inhibitors of cysteine protease inhibition. Accordingly, the compounds disclosed herein may be employed in the treatment of parasitic disease states such as malaria, leishmaniasis and trypanosomiasis (e.g., Chagas' disease) as inhibitors of parasitic cysteine proteases, including the cathepsin-L like cysteine proteases (e.g., cruzain). Moreover, the compounds disclosed herein also find use in the treatment of other mammalian disorders (e.g., cancer and inflammatory disorders) as inhibitors of related mammalian cysteine proteases, including cathepsin L, cathepsin B, cathepsin H, cathepsin K and cathepsin S.

The compounds described herein are potent and selective inhibitors of cathepsin. As a consequence of this activity, the compounds can be used in a variety of in vitro, in vivo and ex vivo contexts to inhibit cathepsin activity.

In one implementation, the method further comprises contacting the cathepsin with the compound in a cell. In another implementation, said contacting occurs in vivo. In another implementation, said contacting occurs in vitro.

In another implementation, the present disclosure provides a method of treating a disorder mediated by a cathepsin, comprising administering to a patient in need thereof a therapeutically effective amount of a compound effective to treat the disorder wherein the compound is a compound of Formula I.

In yet another implementation, the disorder mediated by a cathepsin is a cancer where a cathepsin such as cathepsin K or cathepsin L is upregulated, such as cancers of both epithelial and mesenchymal origin including breast, brain, lung, gastrointestinal, pancreatic, colorectal, melanoma, and head and neck cancers among others. The present compounds also may have a therapeutic effect in tumors such as T cell leukemia, thymoma, T and B cell lymphoma (such as diffuse large B cell lymphoma or transformed (CD20+) indolent lymphoma), colon carcinoma, prostate cancer, ovarian cancer (e.g. ovarian epithelial or primary peritoneal carcinoma) and lung carcinoma (e.g., non-small cell lung cancer or small-cell lung cancer).

Pharmaceutical compositions comprising the thiosemicarbazone compounds described herein can be manufactured by means of conventional mixing, dissolving, granulating, dragee-making, levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically.

The thiosemicarbazone compound can be formulated in the pharmaceutical compositions per se, or in the form of a hydrate, solvate, N-oxide or pharmaceutically acceptable salt, as described herein. Typically, such salts are more soluble in aqueous solutions than the corresponding free acids and bases, but salts having lower solubility than the corresponding free acids and bases may also be formed.

In one implementation, the present disclosure provides a pharmaceutical formulation comprising a compound selected from the compounds, as described above.

The compounds can be provided in a variety of formulations and dosages. The compounds can be provided in a pharmaceutically acceptable form including, where the compound can be formulated in the pharmaceutical compositions per se, or in the form of a hydrate, solvate, N-oxide or pharmaceutically acceptable salt, as described herein. Typically, such salts are more soluble in aqueous solutions than the corresponding free acids and bases, but salts having lower solubility than the corresponding free acids and bases may also be formed.

In one implementation, the compounds are provided as non-toxic pharmaceutically acceptable salts, as noted previously. Suitable pharmaceutically acceptable salts of the compounds disclosed herein include acid addition salts such as those formed with hydrochloric acid, fumaric acid, p-toluenesulphonic acid, maleic acid, succinic acid, acetic acid, citric acid, tartaric acid, carbonic acid or phosphoric acid. Salts of amine groups may also comprise quaternary ammonium salts in which the amino nitrogen atom carries a suitable organic group such as an alkyl, alkenyl, alkynyl or aralkyl moiety. Furthermore, where the compounds disclosed herein carry an acidic moiety, suitable pharmaceutically acceptable salts thereof may include metal salts such as alkali metal salts, e.g. sodium or potassium salts; and alkaline earth metal salts, e.g. calcium or magnesium salts.

The pharmaceutically acceptable salts of the compounds disclosed herein can be formed by conventional means, such as by reacting the free base form of the product with one or more equivalents of the appropriate acid in a solvent or medium in which the salt is insoluble, or in a solvent such as water which is removed in vacuum or by freeze drying or by exchanging the anions of an existing salt for another anion on a suitable ion exchange resin.

The compounds disclosed herein include within its scope solvates of the thiosemicarbazone compounds and salts thereof, for example, hydrates.

The thiosemicarbazone compounds may have one or more asymmetric centers, and may accordingly exist both as enantiomers and as diastereomers. It is to be understood that all such isomers and mixtures thereof are encompassed within the scope of the present disclosure.

The thiosemicarbazone compounds can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients and vehicles appropriate for each route of administration. In addition to the treatment of warm-blooded animals such as mice, rats, horses, cattle, sheep, dogs, cats, monkeys, etc., the compounds disclosed herein can be effective in humans.

The pharmaceutical compositions for the administration of the thiosemicarbazone compounds may conveniently be presented in dosage unit form and can be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired therapeutic effect. For example, pharmaceutical compositions of the present disclosure may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) disclosed herein can be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, dextrose solution, etc., before use. To this end, the active compound(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars, films or enteric coatings. Additionally, the pharmaceutical compositions containing the 2,4-substituted pyrmidinediamine as active ingredient in a form suitable for oral use, may also include, for example, troches, lozenges, aqueous or oily suspensions, dispersible powders or granules, emulsions, hard or soft capsules, or syrups or elixirs. Compositions intended for oral use can be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch, or alginic acid); binding agents (e.g. starch, gelatin or acacia); and lubricating agents (e.g. magnesium stearate, stearic acid or talc). The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and U.S. Pat. No. 4,265,874 to form osmotic therapeutic tablets for control release. The pharmaceutical compositions disclosed herein may also be in the form of oil-in-water emulsions.

Liquid preparations for oral administration may take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, Cremophore™ or fractionated vegetable oils); and preservatives (e.g., methyl or propyl p hydroxybenzoates or sorbic acid). The preparations may also contain buffer salts, preservatives, flavoring, coloring and sweetening agents as appropriate.

Preparations for oral administration can be suitably formulated to give controlled release of the active compound, as is well known.

For buccal administration, the compositions may take the form of tablets or lozenges formulated in conventional manner.

For rectal and vaginal routes of administration, the active compound(s) can be formulated as solutions (for retention enemas) suppositories or ointments containing conventional suppository bases such as cocoa butter or other glycerides.

For nasal administration or administration by inhalation or insufflation, the active compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

The pharmaceutical compositions can be in the form of a sterile injectable aqueous or oleagenous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution and isotonic sodium chloride solution. The thiosemicarbazone compounds may also be administered in the form of suppositories for rectal or urethral administration of the drug. In particular implementations, the compounds can be formulated as urethral suppositories, for example, for use in the treatment of fertility conditions, particularly in males, e.g., for the treatment of testicular dysfunction.

According to the present disclosure, thiosemicarbazone compounds can be used for manufacturing a composition or medicament, including medicaments suitable for rectal or urethral administration. The present disclosure also relates to methods for manufacturing compositions including thiosemicarbazone compounds in a form that is suitable for urethral or rectal administration, including suppositories.

For topical use, creams, ointments, jellies, gels, solutions or suspensions, etc., containing the thiosemicarbazone compounds can be employed. In certain implementations, the thiosemicarbazone compounds can be formulated for topical administration with polyethylene glycol (PEG). These formulations may optionally comprise additional pharmaceutically acceptable ingredients such as diluents, stabilizers and/or adjuvants. In particular implementations, the topical formulations are formulated for the treatment of allergic conditions and/or skin conditions including psoriasis, contact dermatitis and atopic dermatitis, among others described herein.

According to the present disclosure, thiosemicarbazone compounds can be used for manufacturing a composition or medicament, including medicaments suitable for topical administration. The present disclosure also relates to methods for manufacturing compositions including thiosemicarbazone compounds in a form that is suitable for topical administration.

According to the present disclosure, thiosemicarbazone compounds can also be delivered by any of a variety of inhalation devices and methods known in the art, including, for example: U.S. Pat. No. 6,241,969; U.S. Pat. No. 6,060,069; U.S. Pat. No. 6,238,647; U.S. Pat. No. 6,335,316; U.S. Pat. No. 5,364,838; U.S. Pat. No. 5,672,581; WO96/32149; WO95/24183; U.S. Pat. No. 5,654,007; U.S. Pat. No. 5,404,871; U.S. Pat. No. 5,672,581; U.S. Pat. No. 5,743,250; U.S. Pat. No. 5,419,315; U.S. Pat. No. 5,558,085; WO98/33480; U.S. Pat. No. 5,364,833; U.S. Pat. No. 5,320,094; U.S. Pat. No. 5,780,014; U.S. Pat. Nos. 5,658,878; 5,518,998; 5,506,203; U.S. Pat. No. 5,661,130; U.S. Pat. No. 5,655,523; U.S. Pat. No. 5,645,051; U.S. Pat. No. 5,622,166; U.S. Pat. No. 5,577,497; U.S. Pat. No. 5,492,112; U.S. Pat. No. 5,327,883; U.S. Pat. No. 5,277,195; U.S. Pat. Pub. No. 20010041190; U.S. Pat. Pub. No. 20020006901; and U.S. Pat. Pub. No. 20020034477.

Included among the devices which can be used to administer particular examples of the thiosemicarbazone compounds are those well-known in the art, such as, metered dose inhalers, liquid nebulizers, dry powder inhalers, sprayers, thermal vaporizers, and the like. Other suitable technology for administration of particular thiosemicarbazone compounds includes electrohydrodynamic aerosolizers.

In addition, the inhalation device is preferably practical, in the sense of being easy to use, small enough to carry conveniently, capable of providing multiple doses, and durable. Some specific examples of commercially available inhalation devices are Turbohaler (Astra, Wilmington, Del.), Rotahaler (Glaxo, Research Triangle Park, N.C.), Diskus (Glaxo, Research Triangle Park, N.C.), the Ultravent nebulizer (Mallinckrodt), the Acorn II nebulizer (Marquest Medical Products, Totowa, N.J.) the Ventolin metered dose inhaler (Glaxo, Research Triangle Park, N.C.), or the like. In one implementation, thiosemicarbazone compounds can be delivered by a dry powder inhaler or a sprayer.

As those skilled in the art will recognize, the formulation of thiosemicarbazone compounds, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of thiosemicarbazone compounds in the aerosol. For example, shorter periods of administration can be used at higher concentrations of thiosemicarbazone compounds in the nebulizer solution. Devices such as metered dose inhalers can produce higher aerosol concentrations, and can be operated for shorter periods to deliver the desired amount of thiosemicarbazone compounds in some implementations. Devices such as dry powder inhalers deliver active agent until a given charge of agent is expelled from the device. In this type of inhaler, the amount of 2 thiosemicarbazone compounds in a given quantity of the powder determines the dose delivered in a single administration. The formulation of thiosemicarbazone is selected to yield the desired particle size in the chosen inhalation device.

Formulations of thiosemicarbazone compounds for administration from a dry powder inhaler may typically include a finely divided dry powder containing thiosemicarbazone compounds, but the powder can also include a bulking agent, buffer, carrier, excipient, another additive, or the like. Additives can be included in a dry powder formulation of thiosemicarbazone compounds, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like.

The present disclosure also relates to a pharmaceutical composition including thiosemicarbazone compounds suitable for administration by inhalation. According to the present disclosure, thiosemicarbazone compounds can be used for manufacturing a composition or medicament, including medicaments suitable for administration by inhalation. The disclosure also relates to methods for manufacturing compositions including thiosemicarbazone compounds in a form that is suitable for administration, including administration by inhalation. For example, a dry powder formulation can be manufactured in several ways, using conventional techniques, such as described in any of the publications mentioned above, and for example, Baker, et al., U.S. Pat. No. 5,700,904. Particles in the size range appropriate for maximal deposition in the lower respiratory tract can be made by micronizing, milling, or the like. And a liquid formulation can be manufactured by dissolving the thiosemicarbazone compounds in a suitable solvent, such as water, at an appropriate pH, including buffers or other excipients.

Pharmaceutical compositions comprising the thiosemicarbazone compounds described herein can be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically.

For ocular administration, the thiosemicarbazone compound(s) can be formulated as a solution, emulsion, suspension, etc. suitable for administration to the eye. A variety of vehicles suitable for administering compounds to the eye are known in the art. Specific non-limiting examples are described in U.S. Pat. No. 6,261,547; U.S. Pat. No. 6,197,934; U.S. Pat. No. 6,056,950; U.S. Pat. No. 5,800,807; U.S. Pat. No. 5,776,445; U.S. Pat. No. 5,698,219; U.S. Pat. No. 5,521,222; U.S. Pat. No. 5,403,841; U.S. Pat. No. 5,077,033; U.S. Pat. No. 4,882,150; and U.S. Pat. No. 4,738,851.

For prolonged delivery, the thiosemicarbazone compound(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are well-known examples of delivery vehicles that can be used to deliver active compound(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device can be accompanied by instructions for administration.

The amount of compound administered will depend upon a variety of factors, including, for example, the particular condition being treated, the mode of administration, the severity of the condition being treated and the age and weight of the patient, the bioavailability of the particular active compound, etc. Determination of an effective dosage is well within the capabilities of those skilled in the art.

As known by those of skill in the art, the preferred dosage of thiosemicarbazone compounds will also depend on the age, weight, general health and severity of the condition of the individual being treated. Dosage may also need to be tailored to the sex of the individual and/or where administered by inhalation, the lung capacity of the individual. Dosage may also be tailored to individuals suffering from more than one condition or those individuals who have additional conditions which affect lung capacity and the ability to breathe normally, for example, emphysema, bronchitis, pneumonia, respiratory infections, etc. Dosage, and frequency of administration of the compounds will also depend on whether the compounds are formulated for treatment of acute episodes of a condition or for the prophylactic treatment of a disorder. For example, acute episodes of allergic conditions, including allergy-related asthma, transplant rejection, etc. A skilled practitioner will be able to determine the optimal dose for a particular individual.

For prophylactic administration, the compound can be administered to a patient at risk of developing one of the previously described conditions. For example, if it is unknown whether a patient is allergic to a particular drug, the compound can be administered prior to administration of the drug to avoid or ameliorate an allergic response to the drug. Alternatively, prophylactic administration can be applied to avoid the onset of symptoms in a patient diagnosed with the underlying disorder. For example, a compound can be administered to an allergy sufferer prior to expected exposure to the allergen. Compounds may also be administered prophylactically to healthy individuals who are repeatedly exposed to agents known to one of the above-described maladies to prevent the onset of the disorder. For example, a compound can be administered to a healthy individual who is repeatedly exposed to an allergen known to induce allergies, such as latex, in an effort to prevent the individual from developing an allergy. Alternatively, a compound can be administered to a patient suffering from asthma prior to partaking in activities which trigger asthma attacks to lessen the severity of, or avoid altogether, an asthmatic episode.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC₅₀ of the particular compound as measured in an in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Suitable animal models of hypersensitivity or allergic reactions are described in Foster, (1995) Allergy 50(21Suppl):6-9, discussion 34-38 and Tumas et al., (2001), J. Allergy Clin. Immunol. 107(6):1025-1033. Suitable animal models of allergic rhinitis are described in Szelenyi et al., (2000), Arzneimittelforschung 50(11):1037-42; Kawaguchi et al., (1994), Clin. Exp. Allergy 24(3):238-244 and Sugimoto et al., (2000), Immunopharmacology 48(1):1-7. Suitable animal models of allergic conjunctivitis are described in Carreras et al., (1993), Br. J. Ophthalmol. 77(8):509-514; Saiga et al., (1992), Ophthalmic Res. 24(1):45-50; and Kunert et al., (2001), Invest. Ophthalmol. Vis. Sci. 42(11):2483-2489. Suitable animal models of systemic mastocytosis are described in O'Keefe et al., (1987), J. Vet. Intern. Med. 1(2):75-80 and Bean-Knudsen et al., (1989), Vet. Pathol. 26(1):90-92. Suitable animal models of hyper IgE syndrome are described in Claman et al., (1990), Clin. Immunol. Immunopathol. 56(1):46-53. Suitable animal models of B-cell lymphoma are described in Hough et al., (1998), Proc. Natl. Acad. Sci. USA 95:13853-13858 and Hakim et al., (1996), J. Immunol. 157(12):5503-5511. Suitable animal models of atopic disorders such as atopic dermatitis, atopic eczema and atopic asthma are described in Chan et al., (2001), J. Invest. Dermatol. 117(4):977-983 and Suto et al., (1999), Int. Arch. Allergy Immunol. 120(Suppl 1):70-75. Suitable animal models of transplant rejection, such as models of HVGR are described in O'Shea et al., (2004), Nature Reviews Drug Discovery 3:555-564; Cetkovic-Curlje & Tibbles, (2004), Current Pharmaceutical Design 10:1767-1784; and Chengelian et al., (2003), Science 302:875-878. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

Also provided are kits for administration of the thiosemicarbazone or pharmaceutical formulations comprising the compound, that may include a dosage amount of at least one thiosemicarbazone or a composition comprising at least one thiosemicarbazone as disclosed herein. Kits may further comprise suitable packaging and/or instructions for use of the compound. Kits may also comprise a means for the delivery of the at least one thiosemicarbazone or compositions comprising at least thiosemicarbazone, such as an inhaler, spray dispenser (e.g. nasal spray), syringe for injection or pressure pack for capsules, tables, suppositories, or other device as described herein.

Additionally, the compounds disclosed herein can be assembled in the form of kits. The kit provides the compound and reagents to prepare a composition for administration. The composition can be in a dry or lyophilized form, or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to syringe, pipette, transdermal patch, or inhalant.

The kits may include other therapeutic compounds for use in conjunction with the compounds described herein. In one implementation, the therapeutic agents are immunosuppressant or anti-allergen compounds. These compounds can be provided in a separate form, or mixed with the compounds disclosed herein.

The kits will include appropriate instructions for preparation and administration of the composition, side effects of the compositions, and any other relevant information. The instructions can be in any suitable format, including, but not limited to, printed matter, videotape, computer readable disk, or optical disc.

In one implementation, the present disclosure provides a kit comprising a compound selected from the compounds disclosed herein, packaging, and instructions for use.

Kits may also be provided that contain sufficient dosages of thiosemicarbazone or composition to provide effective treatment for an individual for an extended period, such as a week, 2 weeks, 3, weeks, 4 weeks, 6 weeks or 8 weeks or more.

It will be appreciated by one of skill in the art that the implementations summarized above may be used together in any suitable combination to generate additional implementations not expressly recited above, and that such implementations are considered to be part of the present disclosure.

### EXAMPLES

The following examples are provided by way of illustration only and not by way of limitation. Those of skill in the art will readily recognize a variety of noncritical parameters that could be changed or modified to yield essentially similar results.

### Example 1—Synthesis of Compound 11 and Compound 27

Scheme 6 illustrates an example for a method to synthesize Compound 11 and Compound 27.

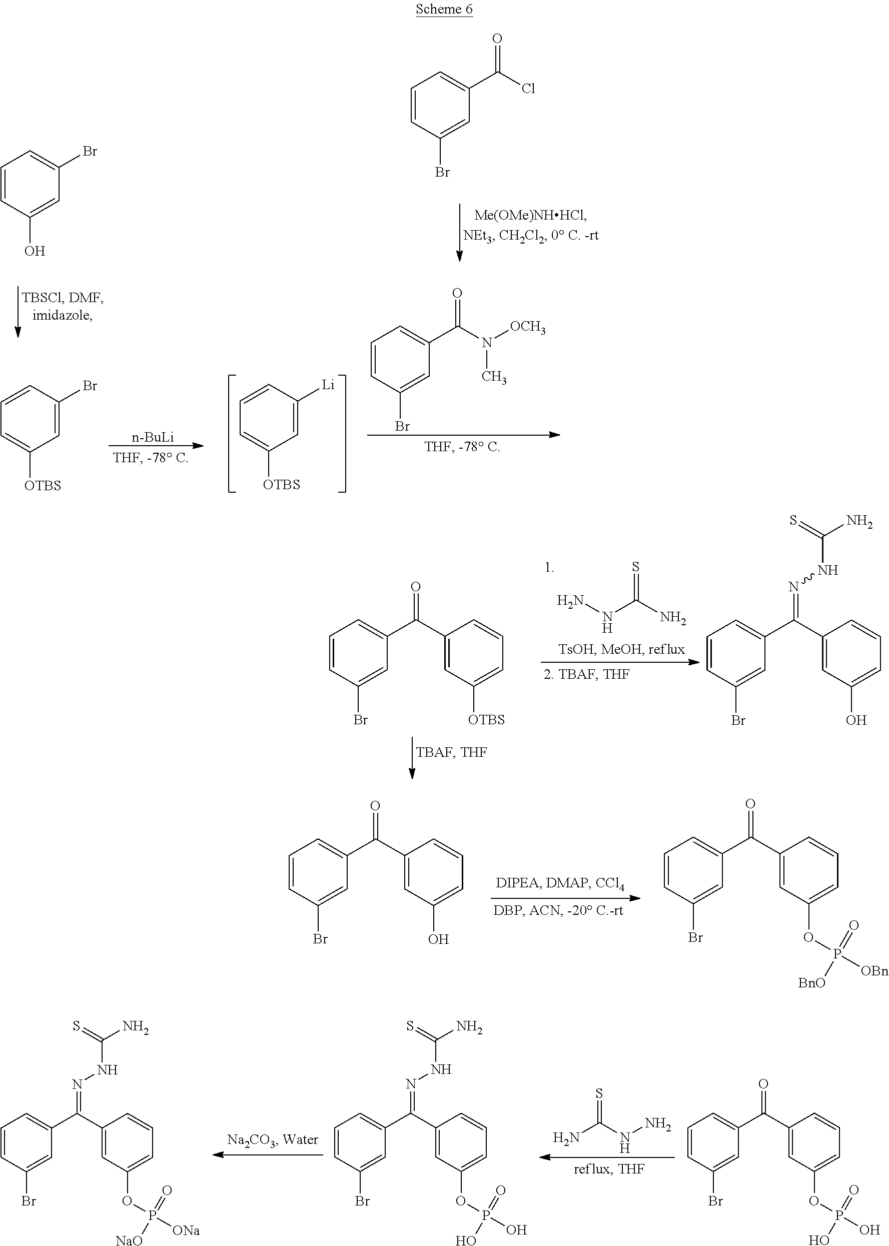

### Synthesis of (3-Bromophenoxy)-tert-butyl-dimethyl-silane

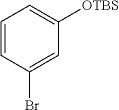 [SMILES: CC(C)(C)[Si](C)(C)Oc1cccc(Br)c1]

Tert-butyl dimethylsilyl chloride (3.150 g, 21.00 mmol) was added to a solution of imidazole (1.900 g, 27.94 mmol) and 3-bromophenol (1.520 mL, 14.01 mmol) in anhydrous DMF (40 mL) at 0° C. The reaction mixture was stirred for 6 hrs. Upon completion of the reaction, 5% aqueous NaHCO₃ (20 ml) was added to the reaction mixture. The products were extracted with hexanes (2×50 mL) and concentrated under reduced pressure. Purification by flash column chromatography (silica gel, hexanes 100%) afforded (3-bromo-phenoxy)-tert-butyl-dimethyl-silane (3.905 g, 13.59 mmol, 97% yield) as a colorless oil. ¹H NMR (500 MHz, CDCl₃) δ 7.10-7.06 (2H, m), 7.01-7.00 (1H, m), 6.78-6.74 (1H, m), 0.98 (9H, s), 0.20 (6H, s). ¹³C NMR (125 MHz, CDCl₃) δ156.67, 130.55, 124.61, 123.66, 122.61, 118.96, 25.75, 18.33, −4.32.

### Synthesis of 3-Bromo-N-methoxy-N-methylbenzamide

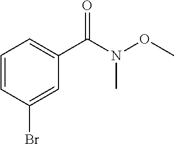

Triethylamine (1919 mL, 13.66 mmol) was added dropwise to a solution of N,O dimethylhydroxylamine hydrochloride (1.000 g, 10.25 mmol) in anhydrous dichloromethane (20 mL) at 0° C. After 10 min of stirring, a solution of 3-bromobenzoyl chloride (902 mL, 6.83 mmol) dichloromethane (5 mL) was added dropwise and the reaction mixture was returned to room temperature. After 4.5 h of stirring, the reaction mixture was quenched with 35 mL of water. The products were extracted with dichloromethane (2×25 mL) and the combined organic phases were dried over anhydrous sodium sulfate and concentrated under reduced pressure. Purification by flash column chromatography (silica gel, hexanes: ethyl acetate, gradient 90:10 to 70:30) afforded 3-bromo-N-methoxy-N-methylbenzamide (1.549 g, 6.83 mmol, 93% yield) as a light yellow oil. ¹H NMR (500 MHz, CDCl₃) δ 7.83 (1H, t, J=1.8 Hz, 1H), 7.62 (1H, dt, J=7.7 Hz, 1.3 Hz), 7.59 (1H, ddd, J=8.0 Hz, 2.0 Hz, 1.0 Hz), 7.28 (1H, t, J=7.9 Hz), 3.55 (3H, s), 3.36 (3H, s). ¹³C NMR (125 MHz, CDCl₃) δ 168.19, 135.93, 133.56, 131.22, 129.61, 126.79, 122.01, 61.20, 33.57.

### Synthesis of [3-(t-Butyldimethylsilyl)oxyphenyl]-(3-bromophenyl) methanone

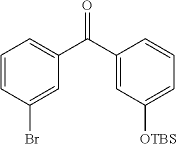 [SMILES: CC(C)(C)[Si](C)(C)Oc1cccc(C(=O)c2cccc(Br)c2)c1]

The (3-bromophenoxy)-tert-butyl-dimethyl-silane (3.905 g, 13.59 mmol) was dissolved in THF (20 mL) and stirred for 5 min. The solution was cooled to −78° C. and stirred for an additional 10 min before dropwise addition of n-butyllithium (2.96 mL, 6.8 mmol). The solution was allowed to stir for 1 hour and 20 minutes. A solution of 3-Bromo-N-methoxy-N-methylbenzamide (1.508 g, 6.17 mmol) in 5 mL THF was added and allowed to stir for 2 hours at −78° C. It is noted that the organo-lithium reagent can be added to the benzamide reagent in a reverse addition protocol. The ice bath was removed and the reaction mixture was allowed to stir for 30 minutes. The reaction mixture was quenched with 20 mL of 1 M HCl, and the organic phase was extracted with chloroform (2×50 mL). The organic phase was washed three times with saturated sodium bicarbonate. The organic phase was separated, dried over sodium sulfate, and concentrated under reduced pressure. Purification by flash column chromatography (silica gel, hexanes:EtOAc, gradient 100:0 to 85:15) afforded [3-(t-Butyldimethylsilyl)oxyphenyl]-(3-bromophenyl)methanone (2.184 g, 5.58 mmol, 91% yield). ¹H NMR (600 MHz, DMSO-d₆) δ 7.89 (1H, ddd, J=8.0 Hz, 2.1 Hz, 1.0 Hz), 7.83 (1H, t, J=1.8 Hz), 7.71 (1H, ddd, J=7.7 Hz, 1.6 Hz, 1.0 Hz), 7.53 (1H, t, J=7.9 Hz), 7.47 (1H, t, J=7.9 Hz), 7.34 (1H, ddd, J=7.6 Hz, 1.6 Hz, 1.0 Hz), 7.20 (1H, ddd, J=8.1 Hz, 2.6 Hz, 1.0 Hz), 7.14 (1H, dd, J=2.5 Hz, 1.6 Hz), 0.95 (s, 9H), 0.20 (s, 6H). ¹³C NMR (150 MHz, DMSO-d₆) δ 193.90, 155.11, 139.16, 137.84, 135.32, 131.84, 130.82, 130.19, 128.56, 124.80, 123.09, 121.80, 120.57, 25.55, 18.01, −4.54.

Another example of a synthesis scheme for the synthesis of [3-(t-Butyldimethylsilyl)oxyphenyl]-(3-bromophenyl) methanone is provided below.

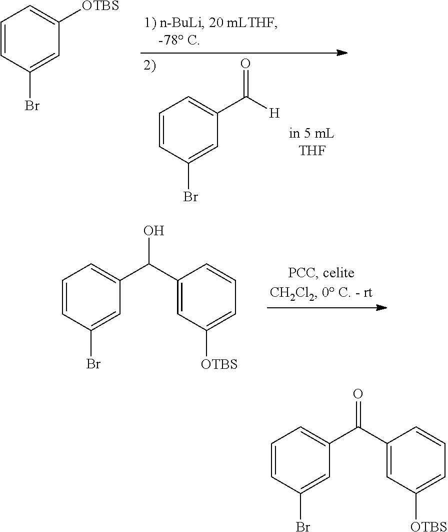 [SMILES: CC(C)(C)[Si](C)(C)Oc1cccc(Br)c1 ; O=Cc1cccc(Br)c1 ; CC(C)(C)[Si](C)(C)Oc1cccc(C(O)c2cccc(Br)c2)c1 ; CC(C)(C)[Si](C)(C)Oc1cccc(C(=O)c2cccc(Br)c2)c1]

### Synthesis of [[3-(t-Butyldimethylsilyl)oxyphenyl]-(3-bromophenyl)-ketone]thiosemicarbazone

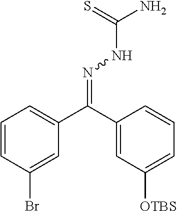 [SMILES: CC(C)(C)[Si](C)(C)Oc1cccc(C(=NNC(N)=S)c2cccc(Br)c2)c1]

[3-(t-Butyldimethylsilyl)oxyphenyl]-(3-bromophenyl) methanone (2.011 g, 5.16 mmol) was dissolved in anhydrous methanol (25 mL), followed by the addition of p-toluenesulfonic acid monohydrate (0.020 g, 105 mmol) and thiosemicarbazone (0.939 g, 10.32 mmol). The reaction mixture was heated to reflux and stirred under an inert atmosphere of nitrogen for 9 h. After reaction completion, methanol was removed under reduced pressure. Products were extracted into EtOAc (2×100 mL) from 100 mL of water. The combined organic phases were washed with brine, dried over anhydrous Na₂SO₄, and concentrated under reduced pressure. Purification by flash column chromatography (silica gel, hexanes:EtOAc, gradient 90:10 to 70:30) afforded [[3-(t-Butyldimethylsilyl)oxyphenyl]-(3-bromophenyl)-ketone] thiosemicarbazone (1.764 g, 3.80 mmol, 73% yield) as a light yellow solid. The above reaction scheme was run several times to generate the materials needed to synthesize compounds 11 and 27. ¹H-NMR (DMSO-d₆, 600 MHz) δ 8.70 (1H, s), 8.57 (1H, s), 8.45 (1H, s), 8.04 (1H, s), 7.59 (1H, ddd, J=8.0 Hz, 2.0 Hz, 1.0 Hz), 7.55 (1H, t, J=7.9 Hz), 7.46 (1H, ddd, J=8.0 Hz, 1.7 Hz, 1.0 Hz) 7.31 (1H, t, J=8.0 Hz), 7.10 (1H, ddd, J=8.3 Hz, 2.5 Hz, 1.0 Hz), 6.94 (1H, ddd, J=7.5 Hz, 1.5 Hz, 1.0 Hz), 6.81 (1H, dd, J=2.5 Hz, 1.5 Hz), 0.945 (9H, s), 0.21 (6H, s). ¹³C-NMR (DMSO-d₆, 150 MHz) δ 177.87, 156.21, 146.80, 138.57, 132.36, 132.03, 131.59, 130.43, 129.46, 126.81, 122.18, 121.87, 121.27, 119.73, 25.59, 18.05, −4.51.

### Synthesis of Compound 11: [(3-Bromophenyl)-(3-hydroxyphenyl)-ketone]thiosemicarbazone

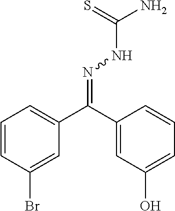 [SMILES: NC(=S)NN=C(c1cccc(O)c1)c1cccc(Br)c1]

[3-(t-Butyldimethylsilyl)oxyphenyl]-(3-bromophenyl) methanone (1.764 g, 3.80 mmol) was dissolved in 30 mL of tetrahydrofuran and tetra-n-butyl ammonium fluoride trihydrate (2.396 g, 7.60 mmol) was added. The reaction mixture was stirred at room temperature under an inert atmosphere of nitrogen gas for 1.5 hrs. After reaction completion, the reaction mixture was diluted with ethyl acetate and washed with brine. The combined organic phases were washed with brine, dried over anhydrous Na₂SO₄, and concentrated under reduced pressure. Purification by flash column chromatography (silica gel, hexanes:EtOAc, gradient 90:10 to 20:80) afforded [(3-Bromophenyl)-(3-hydroxyphenyl)-ketone]thiosemicarbazone (1.070 g, 3.05 mmol, 80% yield) as a light yellow solid. HRMS (ESI) calculated for C₁₄H₁₂BrN₃OSH⁺ [M+H]⁺ 349.99572, found 349.99583. ¹H-NMR (DMSO-d₆, 600 MHz) δ 10.00 (1H, s), 8.70 (1H, s), 8.57 (1H, s), 8.40 (1H, s), 8.10 (1H, s), 7.59 (1H, ddd, J=7.9 Hz, 2.0 Hz, 1.0 Hz), 7.46 (1H, t, J=7.8 Hz), 7.44 (1H, ddd, J=7.9 Hz, 1.7 Hz, 1.0 Hz), 7.31 (1H, t, J=7.9 Hz), 7.00 (1H, ddd, J=8.3 Hz, 2.5 Hz, 1.0 Hz), 6.73 (1H, ddd, J=7.4 Hz, 1.5 Hz, 1.0 Hz), 6.65 (1H, dd, J=2.5 Hz, 1.5 Hz). ¹³C-NMR (DMSO-d₆, 150 MHz) δ 177.76, 158.53, 147.38, 138.51, 132.38, 131.71, 131.42, 130.46, 129.33, 126.98, 122.21, 118.41, 117.24, 114.53.

### Synthesis of (3-Bromophenyl)-(3-hydroxyphenyl) methanone

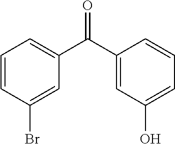 [SMILES: O=C(c1cccc(O)c1)c1cccc(Br)c1]

[3-(t-Butyldimethylsilyl)oxyphenyl]-(3-bromophenyl) methanone (4.857 g, 12.42 mmol) was dissolved in tetrahydrofuran (40 mL) and a solution of tetra-n-butyl ammonium fluoride trihydrate (6.598 g, 16.87 mmol) in THF (10 mL) was added dropwise. The reaction mixture was stirred at room temperature for 45 min. After reaction completion, the reaction mixture was concentrated and extracted with ethyl acetate (3×100 mL) from water (100 mL). The combined organic extracts were dried over Na₂SO₄ and concentrated. Purification by flash column chromatography (silica gel, hexanes:EtOAc, gradient 90:10 to 60:40) afforded (3-bromophenyl)-(3-hydroxyphenyl) methanone (4.07 g, 14.69 mmol, 87% yield) as a light yellow solid. ¹H-NMR (DMSO-d₆, 500 MHz) δ 9.88 (1H, s), 7.87 (1H, ddd, J=8.0 Hz, 2.1 Hz, 1.0 Hz), 7.83 (1H, t, J=1.7 Hz, ArH), 7.69 (1H, ddd, J=7.7 Hz, 1.5 Hz, 1.1 Hz), 7.52 (1H, t, J=7.8 Hz), 7.37 (1H, t, J=7.8 Hz), 7.12-7.15 (2H, m), 7.08 (1H, ddd, J=8.1 Hz, 2.5 Hz, 1.1 Hz). ¹³C-NMR (DMSO-d₆, 125 MHz) 194.24, 157.46, 139.44, 137.57, 135.10, 131.67, 130.73, 129.84, 128.50, 121.79, 120.68, 120.27, 115.96.

### Synthesis of (3-(3-bromobenzoyl)phenyl dibenzyl phosphate

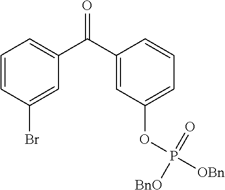 [SMILES: O=C(c1cccc(Br)c1)c1cccc(OP(=O)(OCc2ccccc2)OCc2ccccc2)c1]

(3-Bromophenyl)-(3-hydroxyphenyl) methanone (3.75 g, 13.65 mmol) was dissolved in 50 mL of anhydrous acetonitrile. The flask was cooled to −40° C. using a dry ice/acetonitrile bath followed by the dropwise addition of carbon tetrachloride (6.5 mL, 68.25 mmol) and stirred for 5 min. 4-Dimethylaminopyridine (0.153 g, 1.36 mmol), N,N-diisopropylethylamine (5.00 mL, 28.68 mmol), and dibenzyl phosphite (4.38 mL, 19.79 mmol) were added dropwise. The solution was allowed to slowly come to room temperature and stirred for 1 h 20 min. After reaction completion, water (100 mL) was added to the reaction mixture and products were extracted into EtOAc (3×100 mL). The combined organic phases were dried over anhydrous Na₂SO₄ and concentrated under reduced pressure. Purification by flash column chromatography (silica gel, hexanes:EtOAc, gradient 90:10 to 30:70) afforded (3-(3-bromobenzoyl)phenyl dibenzyl phosphate (6.698 g, 12.47 mmol, 91% yield) as a yellow oil. ³¹P-NMR (DMSO, 85% phosphoric acid as external standard, decoupled, 243 MHz) δ−5.35. ¹H-NMR (DMSO-d₆, 500 MHz) δ 7.91 (1H, ddd, J=8.0 Hz, J=2.1 Hz, J=1.0 Hz), 7.85 (1H, t, J=1.8 Hz), 7.67 (1H, ddd, J=7.7 Hz, 1.6 Hz, 1.0 Hz), 7.61-7.56 (2H, m), 7.53-7.47 (3H, m), 7.36-7.33 (10H, m), 5.19 (4H, d, J=8.8 Hz). ¹³C NMR (DMSO-d₆, 125 Hz) δ 193.15, 150.10 (d, J=6.4 Hz), 138.68, 137.93, 135.50, 135.46 (d, J=6.4 Hz), 131.78, 130.79, 130.45, 128.60, 128.54, 128.46, 128.01, 126.58, 124.60 (d, J=4.8 Hz), 121.93, 120.65 (d, J=4.6 Hz), 69.63, 69.58, ³¹P-NMR (DMSO, decoupled, 202 MHz) δ−6.39.

### Synthesis of 3-(3-bromobenzoyl)phenyl dihydrogen phosphate

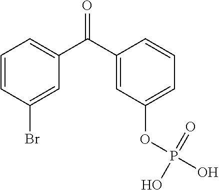 [SMILES: O=C(c1cccc(Br)c1)c1cccc(OP(=O)(O)O)c1]

The phosphate ester (6.58 g, 12.25 mmol) was dissolved 33% HBr in AcOH (25 mL) and the reaction mixture was stirred under air. After 1 h, water (75 mL) was added to the flask (30 mL) and the resulting mixture was washed with hexanes (5×50 mL) at which point the product precipitated out of solution. The aqueous layer was cooled to 0° C. and the solid was filtered and rinsed with ice cold water (15 mL). The solid was allowed to dry overnight in the vacuum filter flask to yield 3-(3-bromobenzoyl)phenyl dihydrogen phosphate (3.839 g, 10.75 mmol, 88%) as a white solid. ¹H NMR (600 MHz, DMSO) δ 11.98 (2H, brs), 7.90 (1H, ddd, J=8.0 Hz, 2.1 Hz, 1.0 Hz), 7.88-7.87 (1H, m), 7.72 (1H, ddd, J=7.7 Hz, 1.6 Hz, 1.0 Hz), 7.58-7.56 (2H, m), 7.54 (1H, t, J=7.84), 7.52-7.50 (1H, m), 7.49-7.46 (1H, m). ¹³C NMR (150 MHz, DMSO) δ 193.59, 151.63 (d, J=6.2 Hz), 138.96, 137.60, 135.46, 131.83, 130.85, 130.10, 128.69, 125.58, 125.00 (d, J=5.2 Hz), 121.98, 120.85 (d, J=4.5 Hz). ³¹P-NMR (DMSO, 85% phosphoric acid as external standard, decoupled, 243 MHz) δ−5.19.

### Synthesis of Compound 27: Disodium (3-bromophenyl)-(3-phosphophenyl)keto thiosemicarbazone

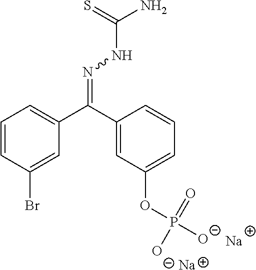 [SMILES: NC(=S)NN=C(c1cccc(Br)c1)c1cccc(OP(=O)([O-])[O-])c1 ; [Na+] ; [Na+]]

A solution of 3-(3-Bromobenzoyl)phenyl dihydrogen phosphate (1.00 g, 2.80 mmol) and thiosemicarbazide (0.510 g, 5.60 mmol) in THF (15 mL) was refluxed for 6 h. After reaction completion (determined from ¹H NMR), the reaction mixture was cooled to room temperature then to 0° C. and the THF layer was carefully transferred to another flask leaving the solid behind. The filtrate was concentrated under a stream of N₂ and further dried under vacuum. The crude product was used without further purification.

Sodium carbonate (0.445 g, 4.20 mmol) was added to a suspension of crude 3-(3-bromobenzoyl)phenyl phosphate thiosemicarbazone in water (5 mL) and allowed to stir for 10 min. The reaction mixture was washed with EtOAc (3×10 mL) and the aqueous layer was concentrated to 2-3 mL using a stream of N₂ gas. After purification by flash column chromatography (C-18, water:acetonitrile, 90:10), the eluent was concentrated under a stream of N₂ gas to 15 mL followed by lyophilization to afford disodium (3-bromophenyl)-(3-phosphophenyl)keto thiosemicarbazone (0.956 g, 2.02 mmol, 72% yield over two steps) as a yellow solid. Purification by reversed-phase thin-layer chromatography followed by concentration of the extracted product under nitrogen resulted in a product that was less soluble in water compared to the purification as described above. Also, at this stage of synthesis and purification, heat was avoided. ¹H NMR (600 MHz, D₂O) δ 7.82 (0.6H, t, J=1.9 Hz), 7.73 (0.4H, ddd, J=8.1 Hz, 2.0 Hz, 1.0 Hz), 7.59-7.54 (2H, m), 7.51-7.47 (1H, m), 7.39 (0.6H, ddt, J=8.3 Hz, 2.3 Hz, 1.1 Hz), 7.34 (0.4H, ddd, J=7.7 Hz, 1.6 Hz, 1.0 Hz), 7.28-7.23 (1.4H, m), 7.07-7.06 (0.6H, m), 7.00-6.98 (0.4H, m), 6.97-6.95 (0.6H, m). ¹³C NMR (150 MHz, D₂O) δ 176.96, 176.76, 154.66 (d, J=6.1 Hz, 153.94 (d, J=6.1 Hz), 152.00, 151.83, 138.35, 137.04, 133.47, 133.16, 133.03, 131.99, 131.37, 131.14, 130.54, 130.42, 130.03, 129.21, 127.49, 126.82, 122.95, 122.86, 122.68 (d, J=4.1 Hz), 122.39, 122.06 (d, J=4.0 Hz), 122.00, 120.46 (d, J=5.2 Hz), 118.74 (d, J=4.8 Hz). ³¹P-NMR (DMSO, 85% phosphoric acid as external standard, decoupled, 243 MHz) δ 0.77, 0.65.

### Example 2—Synthesis of Compound 12

Scheme 7 illustrates an example of a method to synthesize Compound 12.

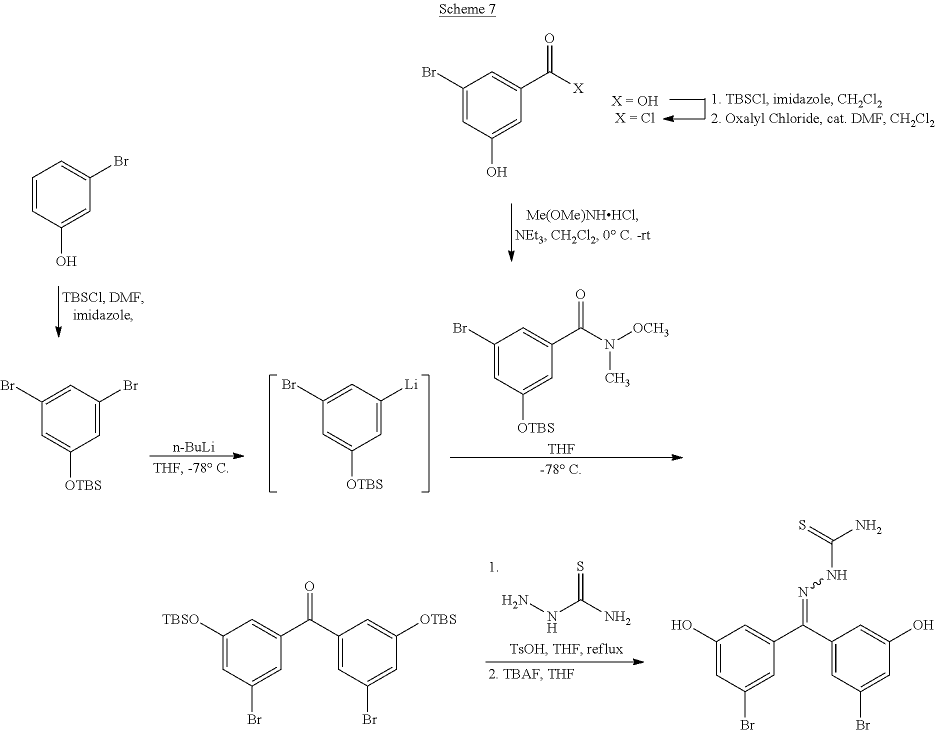

### Synthesis of (3,5-dibromophenoxy)-tert-butyldimethylsilane

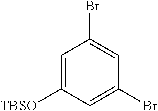 [SMILES: CC(C)(C)[Si](C)(C)Oc1cc(Br)cc(Br)c1]

3,5-dibromophenol (3.78 g, 15.0 mmol) was dissolved in N,N-dimethylformamide (45 mL) followed by the addition of imidazole (2.04 g, 30.0 mmol). The reaction mixture was cooled to 0° C. and tert-butyldimethylchlorosilane (3.37 g, 22.5 mmol) was added. The reaction mixture was returned to room temperature and stirred for 4 h. After reaction completion, the reaction mixture was quenched with saturated aqueous sodium bicarbonate (50 mL) and the product was extracted with hexanes (3×50 mL). The organic extracts were dried over anhydrous sodium sulfate and concentrated under reduced pressure. The crude mixture was purified using flash chromatography (silica gel, hexanes) to afford (3,5-dibromophenoxy)-tert-butyldimethylsilane (5.38 g, 14.7 mmol, 98%). ¹H NMR (600 MHz, CDCl₃): δ 7.26 (1H, t, J=1.7 Hz), 6.93 (2H, d, J=1.7 Hz), 0.97 (9H, s), 0.21 (6H, s). ¹³C NMR (150 MHz, CDCl₃): δ 156.98, 127.12, 122.76, 122.35, 25.49, 18.12, −4.53.

### Synthesis of tert-butyldimethylsilyl 3-bromo-5-((tert-butyldimethylsilyl)oxy) benzoate

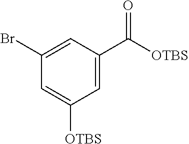 [SMILES: CC(C)(C)[Si](C)(C)OC(=O)c1cc(Br)cc(O[Si](C)(C)C(C)(C)C)c1]

3-bromo-5-hydroxybenzoic acid (1.960 g, 9.031 mmol) and imidazole (4.064, 27.09 mmol) were dissolved in anhydrous dichloromethane (35 mL) followed by the addition of tert-butyldimethylchlorosilane (3.684, 54.18 mmol) and the reaction mixture was refluxed for 5 h. The reaction mixture was allowed to cool to room temperature and was quenched with water (50 mL) and the product was extracted with dichloromethane (3×50 mL). The organic extracts were dried over sodium sulfate and concentrated. The crude mixture was purified using flash chromatography (silica gel, hexanes:ethyl acetate, gradient, 98:02 to 70:30) to afford tert-butyldimethylsilyl 3-bromo-5-((tert-butyldimethylsilyl)oxy)benzoate (1.587 g, 3.562 mmol, 39%). ¹H NMR (600 MHz, CDCl₃): δ 7.84 (1H, t, J=1.6 Hz), 7.47 (1H, dd, J=2.3 Hz, 1.4 Hz), 7.23 (1H, dd, J=2.3 Hz, 1.8 Hz), 0.99 (9H, s), 0.91 (9H, s), 0.24 (6H, s), 0.11 (6H, s). ¹³C NMR (150 MHz, CDCl₃): δ 169.89, 156.54, 131.71, 128.66, 126.11, 122.56, 120.35, 25.62, 25.54, 18.17, 17.98, −3.60, −4.49.

### Synthesis of 3-bromo-5-((tert-butyldimethylsilyl)oxy)benzoyl chloride

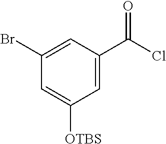 [SMILES: CC(C)(C)[Si](C)(C)Oc1cc(Br)cc(C(=O)Cl)c1]

Oxalyl chloride (359 μL, 4.19 mmol) was added dropwise to a solution of tert-butyldimethylsilyl 3-bromo-5-((tert-butyldimethylsilyl)oxy)benzoate (1.494 g, 3.353 mmol) in dichloromethane (10 mL). A catalytic amount of DMF (2.6 μL, 0.034 mmol) was added dropwise and the reaction mixture was stirred for 12 h at room temperature. After concentration, the reaction mixture was dissolved in dichloromethane and concentrated and repeated to afford 3-bromo-5-((tert-butyldimethylsilyl)oxy)benzoyl chloride. The product was used immediately without further purification. ¹H NMR (600 MHz, CDCl₃): δ 7.85 (1H, t, J=1.7 Hz), 7.47 (1H, dd, J=2.3 Hz, 1.7 Hz), 7.29 (1H, dd, J=2.3 Hz, 1.7 Hz), 0.99 (9H, s), 0.25 (6H, s). ¹³C NMR (150 MHz, CDCl₃): δ 167.01, 156.77, 135.53, 130.03, 127.05, 122.99, 121.21, 25.50, 18.18, −4.49.

### Synthesis of 3-bromo-5-((tert-butyldimethylsilyl)oxy)-N-methoxy-N-methylbenzamide

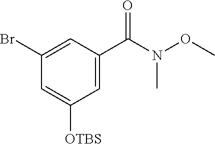 [SMILES: CON(C)C(=O)c1cc(Br)cc(O[Si](C)(C)C(C)(C)C)c1]

Triethylamine (0.941 mL, 6.70 mmol) was added dropwise to a solution of N,O-dimethylhydroxylamine hydrochloride (0.512 g, 5.03 mmol) in dichloromethane (12 mL) at 0° C. A solution of 3-bromo-5-((tert-butyldimethylsilyl)oxy)benzoyl chloride (11.7 g, 3.35 mmol) in dichloromethane (3 mL) was added dropwise to the reaction mixture and the ice bath was removed. After 3 h, the reaction was quenched with water (50 mL) and the product was extracted using dichloromethane (3×20 mL). The combined organic phases were dried over sodium sulfate and concentrated. Purification using flash chromatography (silica gel, hexanes:ethyl acetate, gradient 90:10 to 40:60) afforded 3-bromo-5-((tert-butyldimethylsilyl)oxy)-N-methoxy-N-methylbenzamide (1.10 g, 2.94 mmol, 88% yield over two steps). ¹H NMR (600 MHz, CDCl₃): δ 7.40 (1H, t, J=1.6 Hz), 7.08 (1H, dd, J=2.3 Hz, 1.8 Hz), 7.07-7.06 (1H, m), 3.55 (3H, s), 3.34 (3H, s), 0.98 (9H, s), 0.21 (6H, s). ¹³C NMR (150 MHz, CDCl₃): δ 167.91, 156.00, 136.44, 125.45, 124.15, 122.06, 118.71, 61.21, 25.55, 18.16, −4.49.

### Synthesis of bis(3-bromo-5-((tert-butyldimethylsilyl)oxy)phenyl) methanone

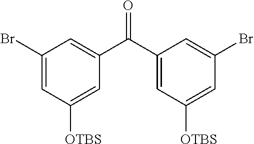 [SMILES: CC(C)(C)[Si](C)(C)Oc1cc(Br)cc(C(=O)c2cc(Br)cc(O[Si](C)(C)C(C)(C)C)c2)c1]

(3,5-Dibromophenoxy)-tert-butyldimethylsilane (1.864 g, 5.091 mmol) was dissolved in THF (12 mL) followed by the addition of n-butyllithium (1.6 M, 1.43 mL) at −78° C. After 1 h, a solution of 3-bromo-5-((tert-butyldimethylsilyl)oxy)-N-methoxy-N-methylbenzamide (0.953 g, 2.55 mmol) in THF (15 mL) was added dropwise to the reaction mixture. After 3 h, the reaction mixture was quenched with hydrochloric acid (1M, 50 mL) and the products were extracted with dichloromethane (3×50 mL). The combined organic phases were washed with sodium bicarbonate (50 mL), dried over anhydrous sodium sulfate, and concentrated. Purification using flash chromatography (silica gel, hexanes: ethyl acetate, gradient 97:3 to 90:10) afforded bis(3-bromo-5-((tert-butyldimethylsilyl)oxy)phenyl) methanone (0.930 g, 1.55 mmol, 67%). ¹H NMR (600 MHz, CDCl₃): δ 7.48 (2H, t, J=1.6 Hz), 7.23 (2H, dd, J=2.3 Hz, 1.8 Hz), 7.13 (2H, dd, J=2.3 Hz, 1.4 Hz), 0.98 (18H, s), 0.23 (12H, s). ¹³C NMR (150 MHz, CDCl₃): δ 192.24, 155.59, 138.47, 126.72, 124.95, 121.82, 119.24, 76.37, 76.16, 75.95, 24.70, 17.33, −5.28.

### Synthesis of [Bis(3-bromo-5-((tert-butyldimethylsilyl)ox)phenyl) ketone]thiosemicarbazone

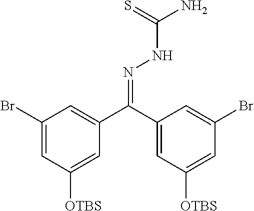 [SMILES: CC(C)(C)[Si](C)(C)Oc1cc(Br)cc(C(=NNC(N)=S)c2cc(Br)cc(O[Si](C)(C)C(C)(C)C)c2)c1]

Bis(3-bromo-5-((tert-butyldimethylsilyl)oxy)phenyl) methanone (0.150 g, 0.250 mmol), thiosemicarbazide (0.0455 g, 0.500 mmol), and p-toluenesulfonic acid monohydrate (0.006 g, 0.03 mmol) were dissolved in anhydrous tetrahydrofuran (5.0 mL) and refluxed for 27 h. The solvent was removed under reduced pressure. Purification using flash chromatography (silica gel, hexanes:ethyl acetate, gradient 99:01 to 80:20) afforded [bis(3-bromo-5-((tert-butyldimethylsilyl)oxy)phenyl) ketone]thiosemicarbazone (0.151 g, 0.224 mmol) in a 90% yield. ¹H NMR (600 MHz, CDCl₃): δ 8.82 (1H, s), 8.21 (1H, s), 7.79 (1H, s), 7.73 (1H, t, J=1.6 Hz), 7.31 (1H, t, J=2.0 Hz), 7.22 (1H, t, J=1.6 Hz), 7.10 (1H, t, J=2.0 Hz), 7.00 (1H, t, J=1.8 Hz), 6.79 (1H, t, J=1.8 Hz), 1.01 (9H, s), 0.94 (9H, s), 0.30 (6H, s), 0.17 (6H, s). ¹³C NMR (150 MHz, CDCl₃): δ 180.63, 158.63, 157.30, 146.00, 140.40, 135.16, 125.87, 125.21, 124.89, 124.68, 123.44, 123.32, 120.21, 119.87, 25.94, 25.93, 18.82, 18.80, −4.37, −4.39.

### Synthesis of Compound 12: [Bis(3-bromo-5-hydroxy) ketone] thiosemicarbazone

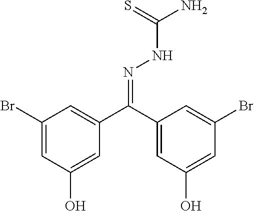 [SMILES: NC(=S)NN=C(c1cc(O)cc(Br)c1)c1cc(O)cc(Br)c1]

Tetra-n-butylammonium fluoride (0.232 g, 0.736 mmol) was added to a solution of [bis(3-bromo-5-((tert-butyldimethylsilyl)oxy)phenyl) ketone] thiosemicarbazone (0.124 g, 0.184 mmol) in tetrahydrofuran (3 mL) and the reaction mixture was allowed to stir for 50 min. The solvent was removed under reduced pressure and the product was extracted from water (10 mL) with ethyl acetate (3×10 mL). The combined organic phases were dried over sodium sulfate and concentrated. Purification using flash chromatography (silica gel, dichloromethane:methanol, gradient 98:02 to 85:15) afforded [bis(3-bromo-5-hydroxy) ketone] thiosemicarbazone (0.0703 g, 0.158 mmol) in a 86% yield. ¹H NMR (600 MHz, Acetone-d₆): δ 9.14 (2H, brs), 8.75 (1H, brs), 8.24 (1H, brs), 7.80 (1H, brs), 7.50 (1H, t, J=1.6 Hz), 7.26 (1H, t, J=2.0 Hz), 7.06-7.04 (2H, m), 6.89 (1H, dd, J=2.3 Hz, 1.5 Hz), 6.85 (1H, dd, J=2.2 Hz, 1.3 Hz). ¹³C NMR (150 MHz, Acetone-d₆): δ 180.55, 160.39, 159.08, 146.66, 140.37, 135.21, 124.76, 123.32, 122.87, 121.72, 121.07, 120.26, 115.35, 115.17. HRMS (ESI) calculated for C₁₄H₁₀Br₂N₃O₂S⁻ [M−H]⁻ 441.88660, found 441.88740.

### Example 3—Synthesis of Compound 13

Scheme 8 illustrates an example of a method to synthesize Compound 13.

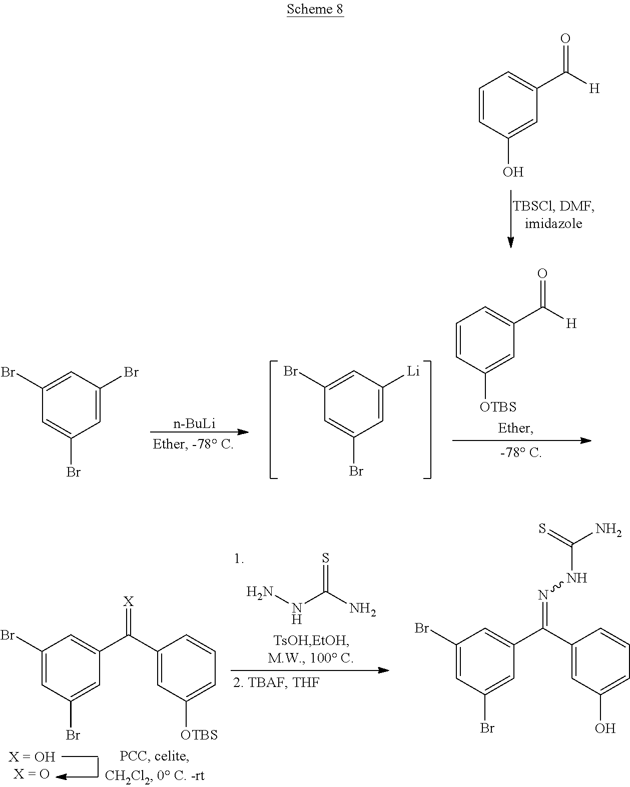

### Synthesis of 3-((tert-butyldimethylsilyl)oxy) benzaldehyde

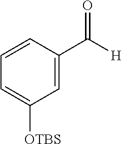 [SMILES: CC(C)(C)[Si](C)(C)Oc1cccc(C=O)c1]

3-hydroxybenzaldehyde (2.000 g, 16.38 mmol) was dissolved in N,N-dimethylformamide (50 mL) followed by the addition of imidazole (2.227 g, 32.75 mmol). The reaction mixture was cooled to 0° C. and tert-butyldimethylchlorosilane (3.684 g, 24.56 mmol) was added. The reaction mixture was returned to room temperature and stirred for 4 h. After reaction completion, the reaction mixture was quenched with saturated aqueous sodium bicarbonate (50 mL) and the product was extracted with hexanes (2×50 mL). The organic extracts were dried over anhydrous sodium sulfate and concentrated under reduced pressure. The crude mixture was purified using flash chromatography (silica gel, hexanes:ethyl acetate, gradient, 100:00 to 90:10) to afford 3-((tert-Butyldimethylsilyl)oxy) benzaldehyde (3.568 g, 15.09 mmol, 92% yield). ¹H NMR (500 MHz, CDCl₃): δ 9.95 (1H, s), 7.47 (1H, dt, J=7.3 Hz, 1.2 Hz), 7.40 (1H, t, J=7.8 Hz), 7.34-7.32 (1H, m), 7.12-7.09 (1H, m), 1.0 (9H, m), 0.23 (6H, m). ¹³C NMR (125 MHz, CDCl₃): δ 192.23, 156.54, 138.07, 130.21, 126.68, 123.69, 120.01, 25.76, 18.34, −4.28.

### Synthesis of 3-((t-Butyldimethylsilyl)oxy)phenyl)-(3,5-dibromophenyl) methanol

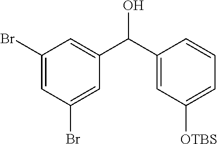 [SMILES: CC(C)(C)[Si](C)(C)Oc1cccc(C(O)c2cc(Br)cc(Br)c2)c1]

Tert-butyllithium (1.7 M, 13.24 mL) was added dropwise to a solution of 1,3,5-tribromobenzene (1.77 g, 17.88 mmol) in diethyl ether (100 mL) at −78° C. The reaction mixture was sonicated for 1 min at 30 min intervals. After 1.5 h, a solution of 3-((tert-butyldimethylsilyl)oxy) benzaldehyde (2.660 g, 11.25 mmol) in diethyl ether (10 mL) was added dropwise to the reaction mixture and stirred at −78° C. After 2 h, the dry ice bath was removed and the reaction mixture was stirred for 18 h. The reaction was quenched with 100 mL of water and extracted with ethyl acetate (2×50 mL) followed by dichloromethane (2×50 mL). The combined organic extracts were dried over sodium sulfate and concentrated. Purification using flash chromatography (silica gel, hexanes:ethyl acetate, gradient 90:10 to 40:60) afforded (3-Bromophenyl)-(3,5-methoxyphenyl) methanol (4.047 g, 8.57 mmol) in a 76% yield. ¹H NMR (600 MHz, CDCl₃): δ 7.55 (1H, t, J=1.8 Hz), 7.46 (2H, dd, J=1.8, 0.7 Hz), 7.22 (1H, t, J=7.8), 6.91 (1H, m), 6.82 (1H, t, J=2.1 Hz), 6.78 (1H, ddd, J=8.1, 2.5, 1.0 Hz), 5.69 (1H, s), 2.26 (1H, d, J=3.1 Hz). ¹³C NMR (150 MHz, CDCl₃): δ 156.03, 147.36, 144.10, 132.97, 129.87, 128.21, 122.93, 119.90, 119.45, 188.29, 74.85, 25.66, 18.23, −4.40.

### Synthesis of 3-((t-Butyldimethylsilyl)oxy)phenyl)-(3,5-dibromophenyl) methanone

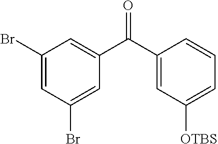 [SMILES: CC(C)(C)[Si](C)(C)Oc1cccc(C(=O)c2cc(Br)cc(Br)c2)c1]

A solution of 3-(t-butyldimethylsilyl)oxyphenyl)-(3,5-dibromophenyl) methanol (3.624 g, 7.673 mmol) in dichloromethane (10 mL) was added dropwise to a suspension of pyridinium chlorochromate (2.474 g, 11.51 mmol) and celite (2.5 g) in anhydrous dichloromethane (40 mL) at 0° C. The ice bath was removed and the reaction was stirred at room temperature. After 5 h, the reaction mixture was filtered over a pad of celite and rinsed with dichloromethane (5×30 mL). The solvent was removed under reduced pressure and purified using flash chromatography (silica gel, hexanes:ethyl acetate, gradient 100:00 to 90:10) afford 3-(t-Butyldimethylsilyl)oxyphenyl)-(3,5-dibromophenyl) methanone (3.523 g, 7.491 mmol) in a 98% yield. ¹H NMR (500 MHz, CDCl₃): δ 7.88 (1H, t, J=1.8 Hz), 7.84 (1H, d, J=1.8 Hz), 7.37 (1H, td, J=7.7, 0.5 Hz), 7.33 (H, dt, J=7.7, 1.5 Hz), 7.22 (1H, ddd, J=2.5, 1.5, 0.5 Hz), 7.10 (1H, ddd, J=7.7, 2.5, 1.5 Hz), 1.00 (s, 9H), 0.23 (s, 6H). ¹³C NMR (125 MHz, CDCl₃): δ 193.24, 155.89, 140.67, 137.61, 137.55, 131.47, 129.72, 125.15, 123.03, 121.15, 25.64, 18.24, −4.37.

### Synthesis of [3-((t-Butyldimethylsilyl)oxy)phenyl)-(3,5-dibromophenyl)-ketone]thiosemicarbazone

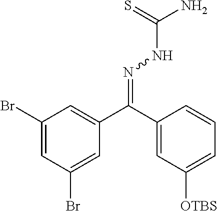 [SMILES: CC(C)(C)[Si](C)(C)Oc1cccc(C(=NNC(N)=S)c2cc(Br)cc(Br)c2)c1]

(3,5-Dibromophenyl)-(3-methoxyphenyl) methanone (0.470 g, 1.00 mmol), thiosemicarbazide (0.455 g, 5.00 mmol), and p-toluenesulfonic acid monohydrate (0.0038 g, 0.020 mmol) were dissolved in anhydrous ethanol (3.0 mL). The reaction was carried out at 100° C. for 2 h under microwave irradiation. The solvent was removed under reduced pressure. Purification using flash chromatography (silica gel, hexanes:ethyl acetate, gradient 95:05 to 60:40) afforded [3-(t-Butyldimethylsilyl)oxyphenyl)-(3,5-dibromophenyl)-ketone] thiosemicarbazone (0.154 g, 0.283 mmol) in a 28% yield. ¹H NMR (600 MHz, Acetone-d₆): δ 9.11 (0.15H, s), 8.63 (0.85H, s), 8.37 (0.85H, s), 8.03 (0.15H, s), 7.99 (0.15H, t, J=1.8 Hz), 7.85 (0.85H, s), 7.81 (1.7H, d, J=1.8 Hz), 7.78 (0.85H, t, J=1.8 Hz), 7.73 (0.15H, s), 7.65 (0.3H, d, J=1.8 Hz), 7.62-7.59 (0.85H, m), 7.34-7.31 (0.15H, m), 7.28 (01.5H, td, J=7.9 Hz, 0.5 Hz), 7.17 (0.85H, ddd, J=8.3 Hz, 2.5 Hz, 1.0 Hz), 7.03 (0.85H, ddd, J=7.5 Hz, 1.5 Hz, 1.0 Hz), 6.98-6.96 (1.0H, m), 6.95 (0.15H, ddd, J=7.9 Hz, 2.5 Hz, 1.1 Hz), 1.00 (7.65H, s), 0.95 (1.35H, s), 0.27 (5.1H, s), 0.17 (0.9H, s). ¹³C NMR (150 MHz, Acetone-d₆): δ 180.45, 157.94, 146.41, 141.60, 135.23, 132.58, 132.52, 129.94, 123.60, 123.28, 122.17, 120.92, 26.02, 18.85, −4.27.

### Synthesis of Compound 13: [(3,5-Dibromophenyl)-(3-hydroxyphenyl) ketone]thiosemicarbazone

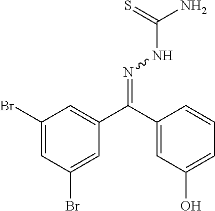 [SMILES: NC(=S)NN=C(c1cccc(O)c1)c1cc(Br)cc(Br)c1]

Tetra-n-butylammonium fluoride (1.0 M in THF, 1.25 mL) was added dropwise to a solution of [3-(t-butyldimethylsilyl)oxyphenyl)-(3,5-dibromophenyl)-ketone] thiosemicarbazone (0.134 g, 0.246 mmol) in tetrahydrofuran (2 mL) and the reaction mixture was allowed to stir for 1.5 h. The solvent was removed under reduced pressure and the product was extracted from water (10 mL) with ethyl acetate(3×10 mL). The combined organic phases were dried over sodium sulfate and concentrated. Purification using flash chromatography (silica gel, hexanes:ethyl acetate, gradient 90:10 to 40:60) afforded [(3-Bromophenyl)-(3,5-methoxyphenyl) ketone] thiosemicarbazone (0.089 g, 0.21 mmol) in a 85% yield. ¹H NMR (600 MHz, DMSO-d₆): δ 10.04 (1H, s), 8.77 (1H, s), 8.71 (1H, s), 8.44 (1H, s), 7.87 (1H, t, J=1.8 Hz), 7.83 (2H, s), 7.48 (1H, t, J=7.9 Hz), 7.01 (1H, ddd, J=8.4, 2.5, 1.0 Hz), 6.74 (1H, dt, J=7.5, 1.3 Hz), 6.68 (1H, t, J=2.0 Hz). ¹³C NMR (150 MHz, DMSO-d₆): δ 177.84, 158.58, 145.83, 140.22, 134.17, 131.56, 131.09, 128.92, 122.77, 118.44, 117.47, 114.54. HRMS (ESI) calculated for C₁₄H₁₁Br₂N₃OSH⁺[M+H]⁺ 427.90623, found 427.90685.

### Example 4—Synthesis of Compound 19 and Compound 21

Scheme 9 illustrates an example of a method to synthesize Compounds 19 and 21.

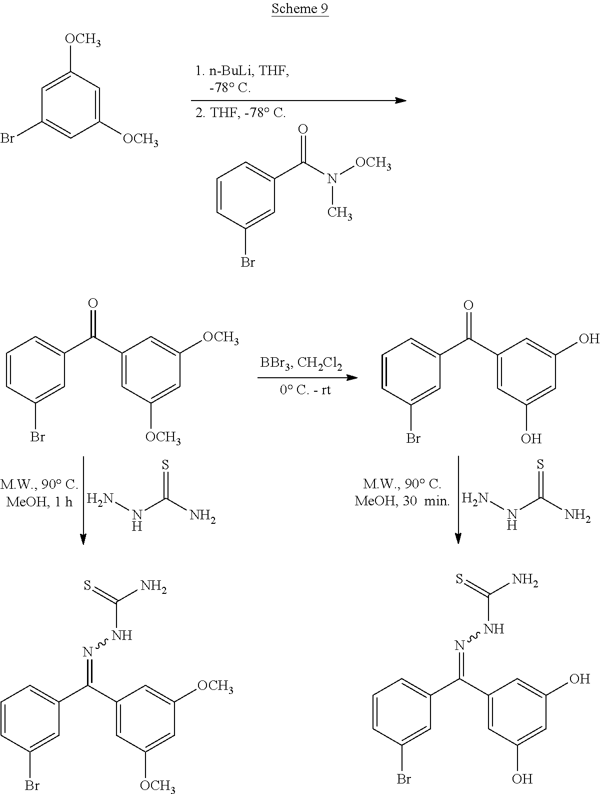

### Synthesis of 3-Bromo-N-methoxy-N-methylbenzamide

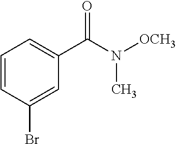

To a well stirred suspension of N,O-dimethylhydroxylamine hydrochloride (5.33 g, 54.7 mmol) in dichloromethane (120 mL) was added triethylamine (10.2 mL, 72.9 mmol) dropwise at 0° C. After stirring for a few minutes, 3-bromobenzoyl chloride (4.81 mL, 36.4 mmol) in dichloromethane (20 mL) was added dropwise. The reaction mixture was allowed to warm to room temperature and stirred for 4 h. The reaction mixture was quenched with water (150 mL) and extracted with dichloromethane (3×100 mL). The combined organic phases were dried over anhydrous sodium sulfate and concentrated. Purification using flash chromatography (silica gel, hexane: ethyl acetate, gradient, 90:10 to 60:40) afforded 3-bromo-N-methoxy-N-methyl-benzamide as a colorless oil (8.604 g, 35.25 mmol, 97% yield). ¹H NMR (500 MHz, CDCl₃): 7.83 (1H, t, J=1.8 Hz), 7.61 (1H, dt, J=7.7 Hz, 1.3 Hz), 7.59 (1H, ddd, J=8.0 Hz, 2.1 Hz, 1.1 Hz), 7.28 (1H, m), 3.35 (3H, s), 3.36 (3H, s). ¹³C NMR (125 MHz, CDCl₃): 168.32, 136.06, 133.69, 131.36, 129.74, 126.93, 122.14, 61.33, 33.71.

### Synthesis of (3-Bromophenyl)-(3,5-dimethoxyphenyl) methanone

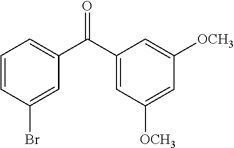 [SMILES: COc1cc(OC)cc(C(=O)c2cccc(Br)c2)c1]

n-Butyllithium in hexanes (2.5 M, 2.88 mL) was added dropwise to a solution of I-bromo-3,5-dimethoxybenzene (2.60 g, 12.0 mmol) in THF (33 mL) cooled to −78° C. After 30 minutes a solution of 3-bromo-N-methoxy-N-methyl-benzamide (1.96 g, 8 mmol) in tetrahydrofuran (7 mL) was added to the reaction mixture and allowed to stir for 2 h at −78° C. After 2 h, the reaction mixture was quenched with water (50 mL) and extracted with dichloromethane (3×50 mL). The combined organic phases were dried over sodium sulfate and concentrated. Purification using flash chromatography (silica gel, hexanes:ethyl acetate, gradient 95:05 to 20:80) (3-bromophenyl)-(3,5-dimethoxyphenyl) methanone (1.97 g, 6.13 mmol) in a 85% yield as a yellow solid. ¹H NMR (600 MHz, CDCl₃): δ 7.95 (1H, t, J=1.8 Hz), 7.71 (2H, dd, J=1.8 Hz), 7.36 (1H, t, J=7.8 Hz), 6.90 (2H, d, J=2.3 Hz), 6.69 (1H, t, J=2.3 Hz), 3.83 (6H, s). ¹³C NMR (150 MHz, CDCl₃): δ 194.84, 160.63, 139.37, 138.71, 135.31, 132.73, 129.81, 128.52, 122.55, 107.84, 105.10, 55.64.

### Synthesis of (3-Bromophenyl)-(3,5-dihydroxyphenyl) methanone

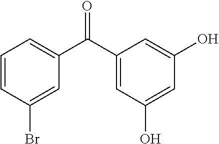 [SMILES: O=C(c1cc(O)cc(O)c1)c1cccc(Br)c1]

(3-Bromophenyl)-(3,5-dimethoxyphenyl) methanone (1.45 g, 4.51 mmol) was dissolved in anhydrous dichloromethane (20 mL) and cooled to 0° C. in an ice bath. Boron tribromide in dichloromethane (1 M, 9.93 mL) was added dropwise to the reaction mixture and the ice bath was removed. After 7 h, boron tribromide in dichloromethane (1 M, 5 mL) was added to the reaction mixture. After an additional 17 h, the reaction mixture was quenched with hydrochloric acid (1M, 40 mL) and the products were extracted with ethyl acetate (3×40 mL). The combined organic phases were washed with sodium bicarbonate (50 mL), dried over anhydrous sodium sulfate, and concentrated. Purification using flash chromatography (silica gel, hexanes: ethyl acetate, gradient 95:5 to 90:10) afforded (3-bromophenyl)-(3,5-dihydroxyphenyl) methanone (0.955 g, 3.26 mmol) in a 72% yield. ¹H NMR (600 MHz, Acetone-d₆): δ 8.70 (2H, s), 7.90 (1H, t, J=1.8 Hz), 7.83 (1H, ddd, J=8.0 Hz, 2.1 Hz, 1.0 Hz), 7.75 (1H, dt, J=7.7 Hz, 1.3 Hz) 7.54 (1H, t, J=7.8 Hz), 6.74 (2H, d, J=2.2 Hz), 7.64 (1H, t, J=2.2 Hz). ¹³C NMR (150 MHz, Acetone-d₆): δ 193.89, 158.59, 140.01, 138.91, 134.93, 132.02, 130.34, 128.44, 121.88, 108.29, 106.95.

### Synthesis of Compound 21: [(3-Bromophenyl)-(3,5-dihydroxyphenyl) ketone]thiosemicarbazide

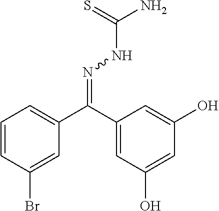 [SMILES: NC(=S)NN=C(c1cc(O)cc(O)c1)c1cccc(Br)c1]

The (3-bromophenyl)-(3,5-dihydroxyphenyl) methanone (0.153 g, 0.52 mmol), thiosemicarbazide (0.0937 mg, 1.03 mmol), p-toluene sulfonic acid monohydrate (0.0054 g, 0.028 mmol) were dissolved in anhydrous methanol (1 mL), sonicated for 30 s, and the reaction was carried out at 90° C. for 30 min under microwave irradiation. The solvent was removed under reduced pressure and the product was extracted from water (5 mL) with ethyl acetate (3×5 mL). The combined organic phases were dried over sodium sulfate and concentrated. Purification was done using flash chromatography (silica gel, hexanes:ethyl acetate, gradient 80:20 to 40:60) afforded [(3-bromophenyl)-(3,5-dihydroxyphenyl) ketone] thiosemicarbazone. ¹H NMR (600 MHz, DMSO-d₆): δ 9.85 (2H, s), 8.70 (1H, s), 8.56 (1H, s), 8.43 (1H, s), 8.11 (1H, s), 7.59 (1H, ddd, J=8.0, 2.0, 1.0 Hz), 7.49 (1H, ddd, J=8.0, 1.7, 1.0 Hz), 7.32 (1H, t, J=8.0 Hz), 6.41 (1H, t, J=2.2 Hz), 6.08 (2H, d, J=2.2 Hz). ¹³C NMR (150 MHz, DMSO-d₆): δ 177.65, 159.90, 147.54, 138.31, 132.38, 132.08, 130.46, 129.27, 126.99, 122.17, 105.44, 103.99. HRMS (ESI) calculated for C₁₄H₁₂BrN₃O₂SH⁺ [M+H]⁺ 359.99064, found 365.99097.

### Synthesis of Compound 19: [(3-Bromophenyl)-(3,5-dimethoxyphenyl) ketone]thiosemicarbazide

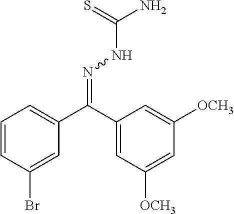 [SMILES: COc1cc(OC)cc(C(=NNC(N)=S)c2cccc(Br)c2)c1]

The (3-Bromophenyl)-(3,5-dimethoxyphenyl) methanone (0.204 g, 0.64 mmol), thiosemicarbazide (0.114 g, 1.25 mmol), p-toluene sulfonic acid monohydrate (0.0059 g, 0.031 mmol) were dissolved in anhydrous methanol (1 mL), sonicated for 30 s, and the reaction was carried out at 90° C. for 1 h under microwave irradiation. The solvent was removed under reduced pressure and the product was extracted from water (5 mL) with ethyl acetate (3×5 mL). The combined organic phases were dried over sodium sulfate and concentrated. Purification using flash chromatography (silica gel, hexanes:ethyl acetate, gradient 90:10 to 40:60) afforded [(3-Bromophenyl)-(3,5-dimethoxyphenyl) ketone]thiosemicarbazone (0.207 g, 0.525 mmol) in a 84% yield. ¹H NMR (600 MHz, DMSO-d₆): δ 8.70 (1H, s), 8.59 (1H, s), 8.44 (1H, s), 8.14 (1H, s), 7.59 (1H, ddd, J=7.9 Hz, 2.0 Hz, 1.0 Hz), 7.45 (1H, ddd, J=7.9 Hz, 1.6 Hz, 1.0 Hz), 7.31 (1H, t, J=8.0 Hz), 6.72 (1H, t, J=2.3 Hz), 6.47 (2H, d, J=2.3 Hz), 3.79 (6H, s). ¹³C NMR (150 MHz, DMSO-d₆): δ 177.82, 161.67, 147.06, 138.35, 132.51, 132.35, 130.45, 129.25, 126.99, 122.21, 105.89, 101.51, 55.59. HRMS (ESI) calculated for C₁₆H₁₅Br₂N₃O₂SNa⁺ [M+Na]⁺ 416.00388, found 416.00403.

### Example 5—Synthesis of Compounds 20 and 22

Scheme 10 illustrates an example of a method to synthesize Compounds 20 and 22.

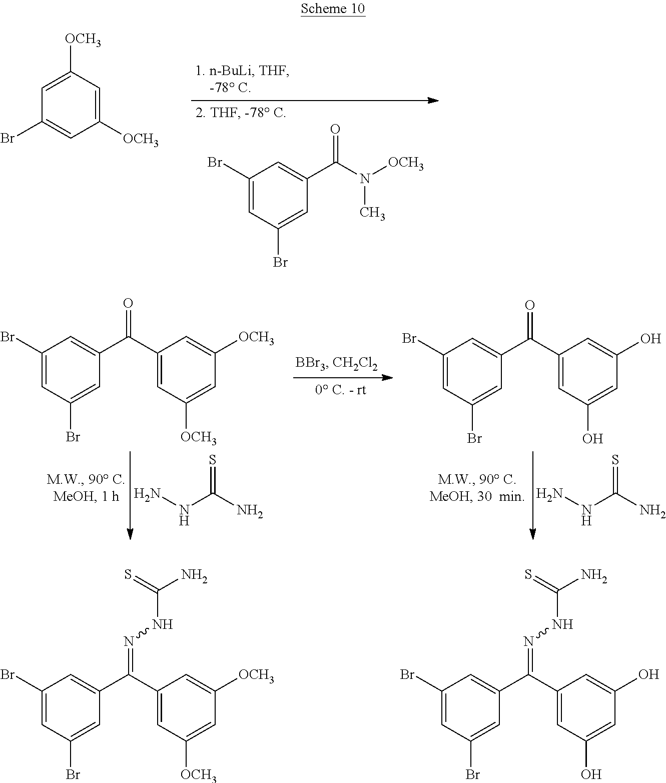

### Synthesis of 3,5-Dibromobenzoyl chloride

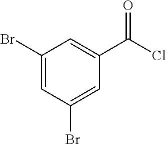 [SMILES: O=C(Cl)c1cc(Br)cc(Br)c1]

Oxalyl Chloride (1.10 mL, 12.9 mmol) was added dropwise to a solution of 3,5-dibromobenzoic acid in anhydrous dichloromethane (50 mL). After 10 min, a catalytic amount of N,N-dimethylformamide (0.0066 μL, 0.086 mmol) was added to the reaction mixture. After 3.5 h, the reaction was concentrated. The product was dissolved in anhydrous dichloromethane (20 mL) and the solvent was removed under reduced pressure. The crude product was used immediately without further purification. ¹H NMR (600 MHz, CDCl₃): δ 8.18 (2H, d, J=1.8 Hz), 7.98 (1H, t, J=1.8 Hz).

### Synthesis of 3,5-Dibromo-N-methoxy-N-methyl-benzamide

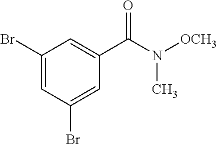

Triethylamine (1.73 mL, 17.1 mmol) was added dropwise to a solution of N,O-dimethylhydroxylamine hydrochloride (1.30 g, 12.9 mmol) in dichloromethane (50 mL) at 0° C. A solution of 3,5-dibromobenzoyl chloride in dichloromethane (10 mL) was added dropwise to the reaction mixture and the ice bath was removed. After 3 h, the reaction was quenched with water (100 mL) and the product was extracted using dichloromethane (3×50 mL). The combined organic phases were dried over sodium sulfate and concentrated. Purification using flash chromatography (silica gel, hexanes:ethyl acetate, gradient 90:10 to 20:80) afforded 3,5-dibromo-N-methoxy-N-methyl-benzamide (2.39 g, 7.40 mmol) in a 86% yield over two steps. ¹H NMR (600 MHz, CDCl₃): δ 7.77-7.75 (3H, m), 3.56 (3H, s), 3.36 (3H, s). ¹³C NMR (150 MHz, CDCl₃): δ 166.59, 137.06, 136.00, 130.02, 122.54, 61.39, 33.30.

### Synthesis of (3,5-Dibromophenyl)-(3,5-dimethoxyphenyl) methanone

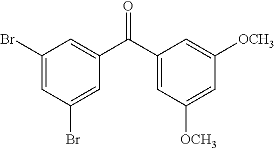 [SMILES: COc1cc(OC)cc(C(=O)c2cc(Br)cc(Br)c2)c1]

To a solution of l-bromo-3,5-dimethoxybenzene (2.28 g, 10.5 mmol) in tetrahydrofuran (33 mL) cooled to −78° C. was added n-butyllithium in hexanes (2.5 M, 2.52 mL) dropwise. After 40 min, a solution of 3,5-dibromo-N-methoxy-N-methyl-benzamide (2.26 g, 8.7 mmol) in tetrahydrofuran (7 mL) was added dropwise and the reaction mixture was allowed to stir for 1.5 h. The reaction was quenched with 1 M HC1 (50 mL) and extracted with dichloromethane (3×50 mL). The combined organic phases were dried over sodium sulfate and concentrated. Purification using flash chromatography (silica gel, hexanes:ethyl acetate, gradient 98:2 to 80:20) afforded (3,5-dibromophenyl)-(3,5-dimethoxyphenyl) methanone (0.935 g, 2.34 mmol) in a 37% yield as a white solid. ¹H NMR (600 MHz, CDCl₃): δ 7.88 (1H, t, J=1.8 Hz), 7.84 (2H, d, J=1.8 Hz), 6.87 (2H, d, J=2.3 Hz), 6.71 (1H, t, J=2.3 Hz), 3.84 (6H, s). ¹³C NMR (150 MHz, CDCl₃): δ 193.39, 160.74, 140.59, 138.06, 137.59, 131.41, 123.02, 107.80, 105.44, 55.68.

### Synthesis of Compound 18: (3,5-dibromophenyl)-(3,5-dihydroxyphenyl) methanone

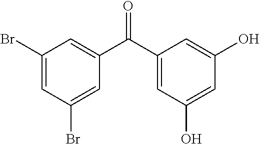 [SMILES: O=C(c1cc(O)cc(O)c1)c1cc(Br)cc(Br)c1]

(3,5-Dibromophenyl)-(3,5-dimethoxyphenyl) methanone (0.606 g, 1.51 mmol) was dissolved in anhydrous dichloromethane (5 mL) and cooled to 0° C. Boron tribromide in dichloromethane (1 M, 3.3 mL) was added dropwise to the reaction mixture and the ice bath was removed. After 7 h, boron tribromide in dichloromethane (1 M, 3.3 mL) was added dropwise to the reaction mixture. After an additional 17 h, the reaction was quenched with 1 M HC1 (20 mL) and the product was extracted with ethyl acetate (3×25 mL). The combined organic extracts were washed with sodium bicarbonate, dried over sodium sulfate and concentrated. Purification using flash chromatography (silica gel, hexanes:ethyl acetate, gradient 88:12 to 0:100) afforded (3,5-dibromophenyl)-(3,5-dihydroxyphenyl) methanone (0.254 g, 0.683 mmol) in a 45% yield. ¹H NMR (600 MHz, Acetone-d₆): δ 8.76 (2H, s), 8.04 (1H, t, J=1.8 Hz), 7.87 (2H, d, J=1.8 Hz), 6.75 (2H, d, J=2.2 Hz) 6.65 (1H, t, J=2.2 Hz). ¹³C NMR (150 MHz, Acetone-d₆): δ 193.35, 159.56, 142.29, 139.12, 137.79, 131.99, 123.47, 109.20, 180.21.

### Synthesis of Compound 22: [(3,5-Dibromophenyl)-(3,5-dihydroxyphenyl) ketone]thiosemicarbazone

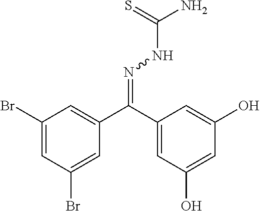 [SMILES: NC(=S)NN=C(c1cc(O)cc(O)c1)c1cc(Br)cc(Br)c1]

(3,5-Dibromophenyl)-(3,5-dihydroxyphenyl) methanone (0.194 g, 0.523 mmol), thiosemicarbazide (0.100 g, 1.10 mmol), and p-toluenesulfonic acid monohydrate (0.049 g, 0.026 mmol) were dissolved in anhydrous methanol (1.5 mL). The reaction was carried out at 90° C. for 30 min under microwave irradiation. The solvent was removed under reduced pressure and the product was extracted from water (5 mL) with ethyl acetate (3×5 mL). The combined organic phases were dried over sodium sulfate and concentrated. Purification using flash chromatography (silica gel, hexanes:ethyl acetate, gradient 80:20 to 40:60) afforded [(3,5-dibromophenyl)-(3,5-dihydroxyphenyl) ketone]thiosemicarbazone (0.163 g, 0.366 mmol) in a 70% yield. ¹H NMR (600 MHz, DMSO-d₆): δ 9.89 (2H, s), 8.76 (1H, s), 8.70 (1H, s), 8.45 (1H, s), 7.90-7.85 (3H, m), 6.43 (1H, t, J=2.2 Hz), 6.09 (2H, d, J=2.2 Hz). ¹³C NMR (150 MHz, DMSO-d₆): δ 177.74, 159.98, 145.99, 140.01, 134.17, 131.47, 128.88, 122.76, 105.44, 104.23. HRMS (ESI) calculated for C₁₄H₁₁Br₂N₃O₂SH⁺ [M+H]⁺ 443.90115, found 443.90132.

### Synthesis of Compound 20: [(3,5-Dibromophenyl)-(3,5-dimethoxyphenyl) ketone]thiosemicarbazone

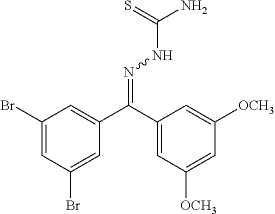 [SMILES: COc1cc(OC)cc(C(=NNC(N)=S)c2cc(Br)cc(Br)c2)c1]

(3,5-Dibromophenyl)-(3,5-dimethoxyphenyl) methanone (0.150 g, 0.375 mmol), thiosemicarbazide (0.0683 g, 0.750 mmol), and p-toluenesulfonic acid monohydrate (0.0035 g, 0.018 mmol) were dissolved in anhydrous methanol (1.0 mL). The reaction was carried out at 90° C. for 1 h under microwave irradiation. The solvent was removed under reduced pressure and the product was extracted from water (15 mL) with ethyl acetate (3×10 mL). The combined organic phases were dried over sodium sulfate and concentrated. Purification using flash chromatography (silica gel, hexanes:ethyl acetate, gradient 93:07 to 50:50) afforded [(3,5-dibromophenyl)-(3,5-dimethoxyphenyl) ketone]thiosemicarbazone (0.120 g, 0.254 mmol) in a 68% yield. ¹H NMR (600 MHz, DMSO-d₆): δ 8.76 (1H, s), 8.72 (1H, s), 8.48 (1H, s), 7.87-7.84 (3H, m), 6.73 (1H, t, J=2.3 Hz), 6.50 (2H, t, J=2.3 Hz), 3.80 (6H, s). ¹³C NMR (150 MHz, DMSO-d₆): δ 177.91, 161.73, 145.55, 140.10, 134.14, 131.88, 128.89, 122.76, 105.93, 101.67, 55.62. HRMS (ESI) calculated for C₁₆H₁₅Br₂N₃O₂SH⁺ [M+H]+ 471.93245, found 471.93283.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A compound of Formula I: 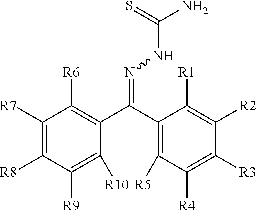
or a solvate or pharmaceutically acceptable salt thereof, wherein
each of R1-R10 are independently selected from the group consisting of: hydrogen, alkoxy, halo, hydroxy, phosphate, phosphate salts, monosodium phosphate, disodium phosphate, dihydrogen phosphate, diphosphate dimer, diphosphate dimer salts, and sodium diphosphate dimers, and at least one of R1-R10 is a phosphate group or diphosphate dimer.

2. The compound of claim 1, wherein at least one of R1-R5 is a phosphate group.

3. The compound of claim 1, wherein at least two of R1-R5 are phosphate groups.

4. The compound of claim 1, wherein at least one of R6-R10 is a phosphate group.

5. The compound of claim 1, wherein at least two of R6-R10 are phosphate groups.

6. The compound of claim 5, wherein R2 and R4 are phosphate groups.

7. The compound of claim 1, wherein one of R1-R5 is a diphosphate dimer group.

8. The compound of claim 1, wherein one of R6-R10 is a diphosphate dimer group.

9. The compound of claim 1, wherein R3 is a phosphate group.

10. The compound of claim 1, wherein R1, R2, R4, and R5 are hydrogen.

11. The compound of claim 1, wherein R4 is a phosphate group.

12. The compound of claim 1, wherein R1-R3 and R5 are hydrogen.

13. The compound of claim 1, wherein the phosphate group is disodium phosphate.

14. The compound of claim 1, wherein the phosphate group is monosodium phosphate.

15. The compound of claim 1, wherein one of R1-R5 is a diphosphate dimer group with one or more sodium atoms.

16. The compound of claim 15, wherein the diphosphate dimer group is a monosodium diphosphate dimer, disodium diphosphate dimer, or trisodium diphosphate dimer.

17. The compound of claim 1, wherein at least one of R6-R10 is a halo.

18. The compound of claim 1, wherein R9 is a halo.

19. The compound of claim 1, wherein R6-R8 and R10 are hydrogen.

20. The compound of claim 1, wherein the halo is bromine (Br).

21. The compound of claim 1, having the formula:

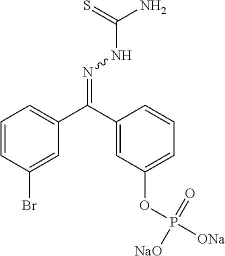

22. The compound of claim 1, wherein R9 is bromine and R4 is monosodium phosphate and R1-R3, R5-R8, and R10 are hydrogen.

23. The compound of claim 1, wherein R9 is bromine and R4 is phosphate and R1-R3, R5-R8, and R10 are hydrogen.

24. A method of inhibiting an activity of a cathepsin, comprising contacting the cathepsin with a compound according to claim 1, in an amount of effective to inhibit an activity of the cathepsin.

25. The method of claim 24, wherein the cathepsin is one or more of: cathepsin B, C, F, H, K, L, O, S, V, W, and X.

26. A method of inhibiting an activity of a cathepsin, comprising contacting in vitro a cathepsin K or cathepsin L with a compound according to claim 1, in an amount effective to inhibit an activity of the cathepsin.

27. A method of inhibiting an activity of a cathepsin, comprising contacting in a patient a cathepsin with a compound according to claim 1, in an amount of effective to inhibit an activity of the cathepsin.

28. The method of claim 24, further comprising: administering a chemotherapy to the patient.

29. The method of claim 24, further comprising: administering a radiation treatment to the patient.

30. A method of inhibiting a neoplasm, comprising administering to a patient suffering from such neoplasm in an amount of a compound according to claim 1 effective to treat the neoplasm.

31. A method of providing an anti-metastatic therapy to a tumor comprising administering to a patient in need of the anti-metastatic therapy a compound according to claim 1.

32. A method of decreasing angiogenesis comprising administering to a patient in need thereof a compound according to claim 1.

33. Use of a compound according to claim 1 to inhibit a neoplasm in a patient suffering from such a neoplasm.

34. A pharmaceutical formulation comprising a compound according to claim 1.

35. A method for synthesizing a compound comprising:
providing a (3-Bromophenoxy)-tert-butyl-dimethyl-silane;
reacting the (3-Bromophenoxy)-tert-butyl-dimethyl-silane with an n-butyllithium to form a (3-lithium-phenoxy)-tert-butyl-dimethyl-silane; and
reacting the (3-lithium-phenoxy)-tert-butyl-dimethyl-silane with a 3-Bromo-N-methoxy-N-methylbenzamide to form a [3-(t-Butyldimethylsilyl)oxyphenyl]-(3-bromophenyl) methanone.

36. The method of claim 35, further comprising reacting the [3-(t-Butyldimethylsilyl)oxyphenyl]-(3-bromophenyl) methanone with a thiosemicarbazide followed by desilylation to form a ([(3-bromophenyl)-(3-hydroxyphenyl)-ketone] thiosemicarbazone).

37. The method of claim 35, further comprising: reacting the [3-(t-Butyldimethylsilyl)oxyphenyl]-(3-bromophenyl) methanone with a tetra-butyl ammonium fluoride trihydrate to form a (3-Bromophenyl)-(3-hydroxyphenyl) methanone.

38. The method of claim 37, further comprising: reacting the (3-Bromophenyl)-(3-hydroxyphenyl) methanone with one or more of: carbon tetrachloride, 4-Dimethylaminopyridine, N,N-diisopropylethylamine, and dibenzyl phosphite to form a dibenzyl (3-(3-bromobenzoyl)phenyl) phosphate.

39. The method of claim 38, further comprising: reacting the dibenzyl (3-(3-bromobenzoyl)phenyl) phosphate with a solution comprising HBr in AcOH or TMSBr to form a 3-(3-bromobenzoyl)phenyl dihydrogen phosphate.

40. The method of claim 39, further comprising: reacting the 3-(3-bromobenzoyl)phenyl dihydrogen phosphate with a thiosemicarbazide followed by reacting with a sodium carbonate to form a 3-(3-bromobenzoyl)phenyl phosphate thiosemicarbazone.
